(19) United States
(12) Patent Application Publication
(10) Pub. No.: US 2009/0087376 A1
(43) Pub. Date: Apr. 2, 2009

(54) Heterocyclic Dye Compounds For In Vivo Imaging And Diagnosis Of Alzheimer's Disease

(75) Inventors: Brian Bacskai, Charlestown, MA (US); Bradley T. Hyman, Swampscott, MA (US); William E. Klunk, Pittsburgh, PA (US); Chester A. Mathis, Pittsburgh, PA (US); Timothy Swager, Newton, MA (US); Evgueni Nesterov, Cambridge, LA (US); Ivory Hills, Harleysville, PA (US)

Correspondence Address: WOLF GREENFIELD & SACKS, P.C., 600 ATLANTIC AVENUE, BOSTON, MA, 02210-2206, US

(73) Assignees: The General Hospital Corporation, Boston, MA (US); Massachusetts Institute of Technology, Cambridge, MA (US); University of Pittsburg of the Commonwealth System of Higher Education, Pittsburg, PA (US)

(21) Appl. No.: 11/632,328

(22) PCT Filed: Jul. 15, 2005
(86) PCT No.: PCT/US2005/025334

§ 371 Date: Feb. 14, 2008

Related U.S. Application Data
(60) Provisional application No. 60/588,281, filed on Jul. 15, 2004.

(51) Int. Cl. A61K 51/04; C07D 249/04; C07D 403/02; C07D 277/62; C07D 263/54; C07D 209/04; A61P 25/00; C07D 409/02; C07D 333/50; A61K 49/00; A61K 49/06

(52) U.S. Cl. 424/1.65; 548/255; 544/300; 548/178; 548/217; 548/468; 549/59; 549/49; 424/9.1; 424/9.3

(57) ABSTRACT

The present invention relates to the identification of compounds that are suitable for imaging amyloid deposits in living patients. The invention relates, in part, to a method of imaging amyloid deposits in brain in vivo to allow antemortem diagnosis of Alzheimer's disease. The present invention also relates to therapeutic uses for such compounds, as exemplified by compounds of the formula (1) in which Y is independently S, O, or N and m is 1, 2, or 3.

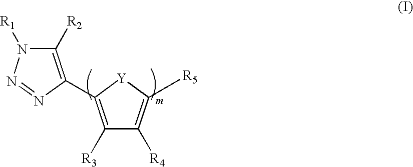

### RELATED APPLICATIONS

This application claims the benefit of U.S. Provisional Patent Application Ser. No. 60/588,281, filed Jul. 15, 2004, the entire contents of which is incorporated by reference herein.

### GOVERNMENT SUPPORT

This invention was made in part with United States government support under grant numbers AG15379 and EB00768 from the National Institutes of Health (NIH). The United States government may have certain rights in this invention.

### FIELD OF THE INVENTION

The present invention relates to the identification of compounds that are suitable for imaging amyloid deposits in living patients. The present invention also relates to methods of imaging amyloid deposits in brain in vivo using such compounds to allow antemortem diagnosis of Alzheimer's disease. The present invention also relates to therapeutic uses for such compounds.

### BACKGROUND OF THE INVENTION

Alzheimer's disease (AD) is a disorder that causes the gradual loss of brain cells. AD is named after Dr. Alois Alzheimer, who in 1906 noticed changes in the brain tissue of a woman who had died of an unusual mental illness. Upon examination, Dr. Alzheimer found abnormal clumps and tangled bundles of fibers, which are now known as amyloid plaques and neurofibrillary tangles, respectively. Today, these plaques and tangles in the brain are considered hallmarks of AD.

AD results in damage in brain regions associated with thought, memory, and language. Symptoms of AD are progressive and include dementia, which includes characteristics such as loss of memory, problems with reasoning or judgment, disorientation, difficulty in learning, loss of language skills, and decline in the ability to perform routine tasks. Additional AD symptoms may include personality changes, agitation, anxiety, delusions, and hallucinations.

The risk of AD in the population increases with age. It is believed that up to 4 million Americans have AD. The onset of AD is generally after age 60, but in rare instances younger individuals may be afflicted. It is generally believed that approximately 3 percent of men and women ages 65 to 74, and almost half of those age 85 and older have AD.

The extracellular plaque formation by amyloid β (Aβ in the brain is an important hallmark in Alzheimer's disease. Associated with the formation of plaque is the transcription of amyloid precursor protein (APP) and the secretion of Aβ. Due in part to the obvious difficulties associated with obtaining brain tissue from living subjects for diagnostic analysis, Alzheimer's disease cannot be diagnosed with certainty until post-mortem. In general, post-mortem assays involve the detection of amyloid-β plaques in harvested brain tissue. No completely effective diagnostic tool is yet available to detect these plaques in a living patient. Due to the lack of suitable diagnostic methods, health-care professionals are only able to provide a tentative diagnosis of AD in an individual, particularly at the early to mid stages of the disease. Although these diagnoses can indicate that a person “likely” has AD, the absence of a definitive diagnosis reflects a critical need for more accurate and reliable AD diagnostic tests.

Amyloid deposits have also been identified in association with various other diseases including Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, inclusion body myositis, multiple sclerosis, and homozygotes for the apolipoprotein E4 allele. (Corder et al., Science, 1993 261: 921). Thus, the development of effective methods to determine the presence of amyloid in tissues and organs of patients would be beneficial for the accurate diagnosis and treatment of these disorders in addition to their use in Alzheimer's disease.

Although recent research has led to the development of contrast agents that should allow direct imaging of these plaques with positron emission tomography (PET), PET imaging is expensive and is not widely available. Thus, the need exists for new in vivo methods to determine the presence of amyloid-β in cells and tissues and to increase the availability of sensitive and reliable methods diagnose to Alzheimer's disease and other amyloid-associated disease.

### SUMMARY OF THE INVENTION

The invention is based in part on the surprising discovery that fluorescent compounds with near infrared (NIR) spectra can be used to target amyloid-β deposits found in Alzheimer's disease and other disorders. The invention also includes, in part, the discovery of novel fluorescent compounds that are useful in the methods of the invention. The NIR spectra permits non-invasive detection of amyloid-β deposits using NIR light and diffuse optical tomography. The compounds include amyloid binding molecules that can be modified for fluorescence in the NIR region, as well as NR fluorescent molecules that can be modified to bind to amyloid-β deposits. The compounds of the invention are designed to access the amyloid-β deposits in the brain. The compounds may be engineered to cross the blood-brain barrier after peripheral injection, or they will be delivered into the cerebral spinal fluid directly. Model NIR fluorescent compounds of the invention are provided.

In addition to the use of the NIR fluorescent compounds of the invention for the assessment of amyloid-β deposits, the compounds are also useful as NIR fluorophores. The NIR compounds of the invention are very small compared to existing NIR fluorophores, and may be used to label proteins or other molecules to track a wide range of targets non-invasively.

According to one aspect of the invention, compounds of the formula:

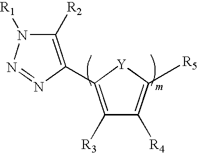

are provided:

wherein R₁-R₄ are independently: X, wherein X is F, Cl, Br or I, (CH₂)ₙOH wherein n=1, 2 or 3, CH₃, CF₃, CN, H, OHX, CHO, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃, SnR′₃ wherein R′ is a lower alkyl group other than CH₃, benzene, substituted benzene, toluene, substituted toluene, xylene, substituted xylene;

R₅ is independently H or an electron withdrawing group: X, wherein X is F, Br, C₁, CF₃, CN, CHO, CONH₂, COOH, COOR′, COCH₃, COR′, NO₂, CON(CH₃)₂, CONR′₂, COOCH₃, COOR′, SO₃H, SO₃R′, CCl₃, NH₄⁺, NR′₃⁺, NR′₄⁺ wherein R′ is a lower alkyl group other than CH₃,

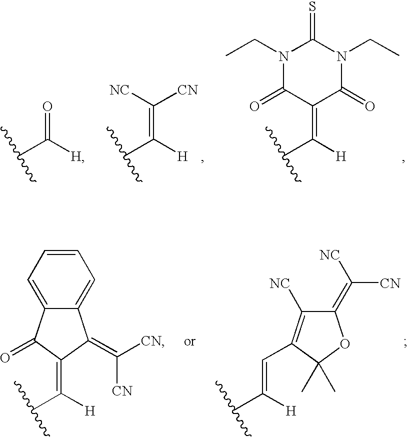

m is 1, 2, or 3; and

Y is independently S, O, or N.

In some embodiments, Y is S. In certain embodiments, R₁ is toluene. In some embodiments of the foregoing compounds, R₂, R₃, and R₄ are H. In some embodiments of the foregoing compounds R₅ is an electron withdrawing group selected from the list of compounds consisting of:

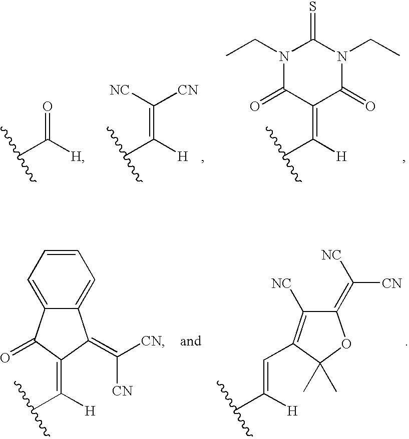

In one embodiment, the compound is:

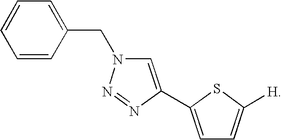

In another embodiment, the compound is:

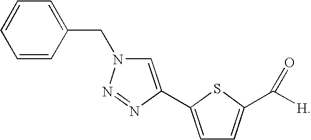

In another embodiment, the compound is:

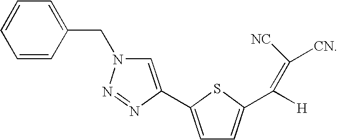

In another embodiment, the compound is:

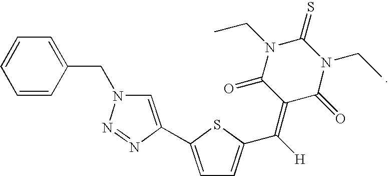

In another embodiment, the compound is:

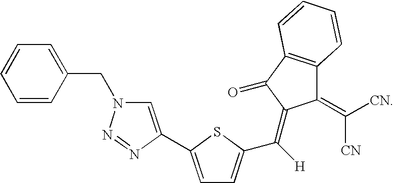

In another embodiment, the compound is:

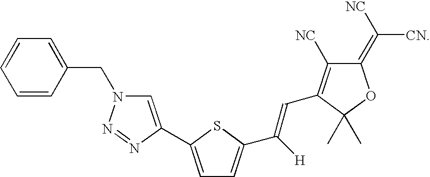

In another embodiment, the compound is:

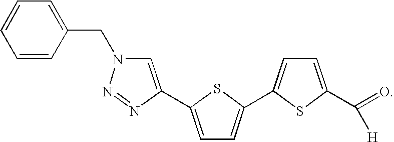

In another embodiment, the compound is:

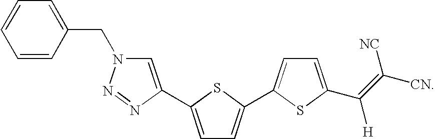

In another embodiment, the compound is:

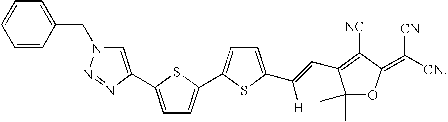

According to one aspect of the invention, compounds of the following formula (compound 1) are provided:

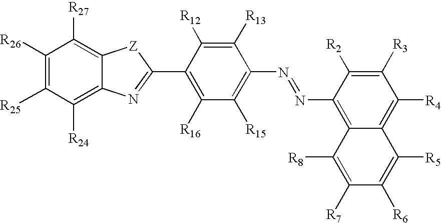

wherein Z is S, NR′, NH or O; and wherein each R₂-R₈, R₁₂, R₁₃, R₁₅, R₁₆ and R₂₄-R₂₇ are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

In some preferred embodiments, R₂, R₄, and R₂₆ are OH. In one preferred embodiment, the compound has the following formula (compound 10, RD1):

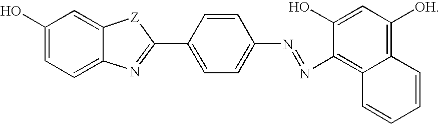

In other preferred embodiments R₂, R₄, R₂₄, and R₂₆ are OH. In one preferred embodiment, the compound has the following formula (compound 11, RD2):

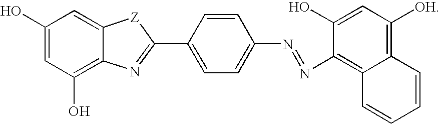

In still other embodiments of compound 1, Z is S; R₅-R₈, R₁₂, R₁₃, R_{15, 16}, R₂₄, R₂₅, and R₂₇ are H; and R₂, R₃, R₄, and R₂₆ are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

In these embodiments, it is preferred that R₂ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₃ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₄ and R₂₆ are H.

Alternatively, it is preferred that R₂ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₂₆ is: X wherein X is F, Cl, Br or I, (CH₂)ₙOH wherein n=1, 2 or 3, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₃ and R₄ are H.

Alternatively, it is preferred that R₃ is X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR₃ wherein R′ is a lower alkyl group other than CH₃; R₄ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₂₆ is: X wherein X is F, Cl, Br or I, (CH₂)ₙOH wherein n=1, 2 or 3, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OR′, SCH₃, SR′, COOCH₃, COOR′, SnCH₃ or SnR′₃ wherein R′ is a lower alkyl group other than CH₃; and R₂ is H.

Alternatively, it is preferred that R₄ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₂₆ is: X wherein X is F, Cl, Br or I, (CH₂)ₙOH wherein n—1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₂ and R₃ are H.

Alternatively, it is preferred that R₂ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₃, R₄ and R₂₆ are H.

Alternatively, it is preferred that R₄ is: X wherein X is F, Cl, Br or I, (CH₂)ₙOH wherein n=1, 2 or 3, CH₃, CF₃, CN, H, HOH, OHX, COH, CONH₂, OCOH, SH, COOH, SnH₃, R′, R′OR′, R′OCH₃, CH₂X, R′X, OCH₂X, OR′X, COCH₃, COR′, N(CH₃)₂, NH₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₂, R₃ and R₂₆ are H.

Alternatively, it is preferred that R₂ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₂₆ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OR′, SCH₃, SR′, COOCH₃, COOR′, SnCH₃ or SnR′₃ wherein R′ is a lower alkyl group other than CH₃; and R₃ and R₄ are H;

Alternatively, it is preferred that R₄ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₂, R₃ and R₂₆ are H.

Alternatively, it is preferred that R₂ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₂₆ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₃ and R₄ are H.

Alternatively, it is preferred that R₄ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂; CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₂₆ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₂ and R₃ are H.

In still other embodiments, R₂ is: X wherein X is F, Cl, Br or I, (CH₂)ₙOH wherein n=1, 2 or 3, CH₃, CF₃, CN, H, HOH, OHX, COH, CONH₂, OCOH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₃ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₄ is: X wherein X is F, Cl, Br or I, (CH₂)ₙOH wherein n=1, 2 or 3, CH₃, CF₃, CN, H, HOH, OHX, COH, CONH₂, OCOH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₂₆ is: X wherein X is F, Cl, Br or I, (CH₂)ₙOH wherein n=1, 2 or 3, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N′R₂, NO₂, CON(CH₃)₂, CONR′₂, OCOCH₃, OCOR′, OR′, SCH₃, SR′, COOCH₃, COOR′, SnCH₃ or SnR′₃ wherein R′ is a lower alkyl group other than CH₃.

In some additional embodiments, the following exclusions to the foregoing compounds apply independently or in combination: if R₂ is OH, R₃ cannot be CO₂H; if R₃ is CO₂H, R₂ cannot be OH; if R₂ is OH, R₂₆ cannot be CH₃; if R₂₆ is CH₃, R₂ cannot be OH; if R₃ is CO₂H, R₄ and R₂₆ cannot be OH and CH₃, respectively; if R₄ is OH, R₃ and R₂₆ cannot be CO₂H and CH₃, respectively; if R₂₆ is CH₃, R₃ and R₄ cannot be CO₂H and OH, respectively; if R₄ is OH, R₂₆ cannot be OCH₃; if R₂₆ is OCH₃, R₄ cannot be OH; R₂ is not NH₂; R₄ is not OH; if R₂ is NH₂, R₂₆ cannot be OCH₃; if R₂₆ is OCH₃, R₂ cannot be NH₂; R₄ is not NH₂; if R₂ is NH₂, R₂₆ cannot be N(CH₃)₂; if R₂₆ is N(CH₃)₂, R₂ cannot be NH₂; if R₄ is OH, R₂₆ cannot be N(CH₃)₂; and if R₂₆ is N(CH₃)₂, R₄ cannot be OH.

In a further set of embodiments of compound 1, Z is O; R₃, R₅-R₉, R₁₂, R₁₃, R₁₅, R₁₆, R₂₄, R₂₆, and R₂₇ are H; and R₂, R₄, and R₂5 are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH3, OCOR′, OCH₃, OR′, SCH₃, SR′, COOCH₃, COOR′, SnCH₃ or SnR′₃ wherein R′ is a lower alkyl group other than CH₃.

In other embodiments of compounds wherein if R₂ is OH, R₃ cannot be CO₂H, R4 is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₂ and R₂₅ are H; or R₂ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; R₂₅ is: X wherein X is F, Cl, Br or I, (CH₂)ₙOH wherein n=1, 2 or 3, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and R₄ is H.

In this further set of embodiments, in certain embodiments the following exclusions apply independently or in combination: R₄ is not NH₂; if R₂ is NH₂, R₂₅ cannot be CH₃; and if R₂₅ is CH₃, R₂ cannot be NH₂.

According to another aspect of the invention, compounds of the following formula (compound 2) are provided:

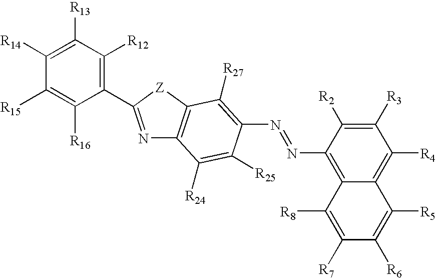

wherein Z is S, NR′, NH or O; and wherein each R₂-R₈, R₁₂-R₁₆ and R₂₄, R₂₅, and R₂₇ are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′; OCH₃, OR′, SCH₃, SR′, COOCH₃, COOR′, SnCH₃ or SnR′₃ wherein R′ is a lower alkyl group other than CH₃.

In some preferred embodiments of compound 2, Z is S; R₂, R₄, R₂₆ are OH; R₂₄ is OH or H; and R₅₋₈, R₁₂, R₁₃, R₁₅, R₁₆, R₂₅ and R₂₇ are H. In other preferred embodiments of compound 2, Z is S; R₂, R₄, R₁₄, are OH; R₁₆ is OH or H; and R₅₋₈, R₁₂₋₁₃, R₁₅ and R₂₄, R₂₅, R₂₇ are H.

According to another aspect of the invention, compounds of the following formula (compound 3) are provided:

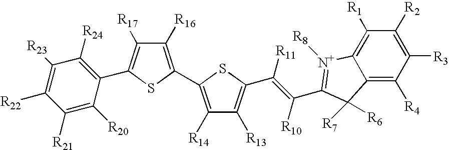

wherein each R₁-R₄, R₆-R₈, and R₁₀-R₁₁, R₁₃-R₁₄, R₁₆-R₁₇, and R₂₀-R₂₄ are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃, and wherein R₈ can be (CH₂)₃SO₂O⁻.

In some embodiments, the compound has the formula in which, R₂₂, R₂₄, and R₂₀ are independently —OH, —NMe₂, —N(CH₂CH₂OH)₂, or hydrogen; there is at least one substituent R₂₂, R₂₄, and R₂₀ different from hydrogen; R₈ is independently Me or —CH₂)₃—SO₂O⁻, with the Me group requiring Cl⁻ as a counteranion; and X is —C(Me₂)—, S, O, or NH.

In preferred embodiments, the compound has the following formula (compound 12, NIAD1):

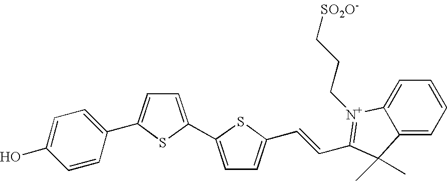

or has the following formula (compound 13, NIAD3):

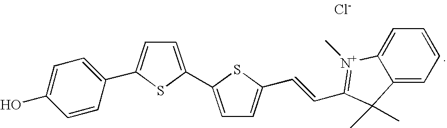

According to still another aspect of the invention, compounds of the following formula (compound 4) are provided:

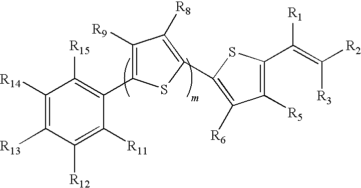

wherein each R₁-R₃, R₅-R₆, and R₈-R₉, and R₁₁-R₁₅, are independently: X wherein X is F, Cl, Br or I, (CH₂)ₙOH wherein n=1, 2 or 3, CH₃, CF₃, CN, H, HOH, OHX, COH, NH₂, CONH₂. OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃, and m is one, two, or three.

In some embodiments, the compound has the formula in which R₁₃, R₁₅, and R₁₁ are independently —OH, —NMe₂, —N(CH₂CH₂OH)₂, or hydrogen; there is at least one substituent R₁₃, R₁₅, and R₁₁ different from hydrogen; and m is one, two, or three. In certain embodiments, the compound has the formula in which R₁₃, R₁₅, and R₁₁ are independently —OH, —NMe₂, —N(CH₂CH₂OH)₂, or hydrogen; there is at least one substituent R₁₃, R₁₅ and R₁₁ different from hydrogen; m can be one, two, or three; and R₁, R₂, and R₃ are CN.

In a preferred embodiment, the compound has the following formula (compound 14, NIAD4):

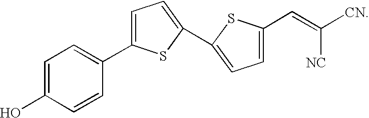

According to a further aspect of the invention, compounds of the following formula (compound 5) are provided:

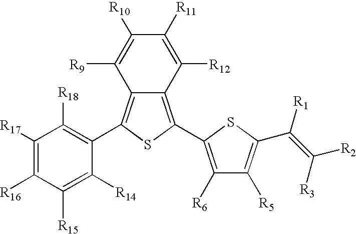

wherein each R₁-R₃, R₅-R₆, and R₉-R₁₂, and R₁₄-R₁₈, are independently: X wherein X is F, Cl, Br or I, (CH₂)ₙOH wherein n=1, 2.0r 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′3 wherein R′ is a lower alkyl group other than CH₃.

In some embodiments, the compound has the formula in which R₁₆, R₁₈, and R₁₄ are independently —OH, —NMe₂, —N(CH₂CH₂OH)₂, or hydrogen; and there is at least one substituent R₁₆, R₁₈, and R₁₄ different from hydrogen.

In a preferred embodiment, the compound has the following formula (compound 15, NIAD6):

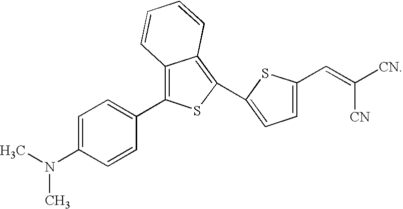

According to a further aspect of the invention, compounds of the following formula (compound 6) are provided:

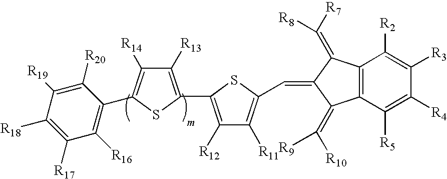

wherein each R₂-R₅, R₇-R₁₀, R₁₁-R₁₄, and R₁₆-R₂₀, are independently: X wherein X is F, Cl, Br or I, (CH₂)ₙOH wherein n—1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃, and m is one, two, or three.

In some embodiments, the compound has the formula in which R₁₈, R₂₀, and R₁₆ are independently —OH, —NMe₂, —N(CH₂CH₂OH)₂, or hydrogen; there is at least one substituent R₁₈, R₂₀, or R₁₆ different from hydrogen; and m one, two, or three.

In a preferred embodiment, the compound has the following formula (compound 16, NIAD7):

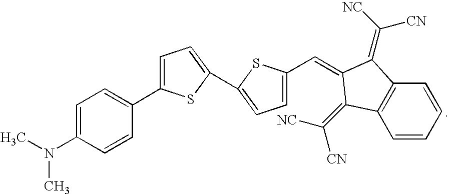

According to a further aspect of the invention, compounds of the following formula (compound 7) are provided:

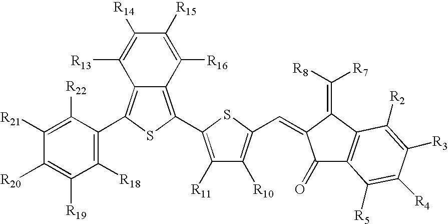

wherein each R₂-R₅, R₇-R₈, R₁₀-R₁₁, R₁₃-R₁₆, and R₁₈-R₂₂, are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

In a preferred embodiment, the compound has the following formula (compound 17, NIAD8):

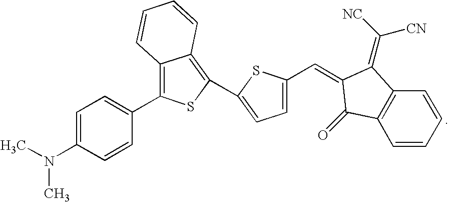

According to a further aspect of the invention, compounds of the following formula (compound 8) are provided:

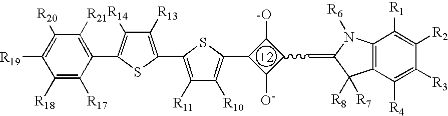

wherein each R₁-R₄, R₆-R₈, R₁₀-R₁₁, R₁₃-R₁₄, and R₁₇-R₂₁, are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

In a preferred embodiment, the compound has the following formula (compound 18, NIAD9):

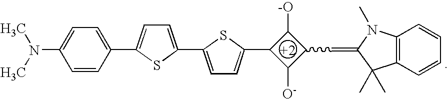

According to a further aspect of the invention, compounds of the following formula (compound 9) are provided:

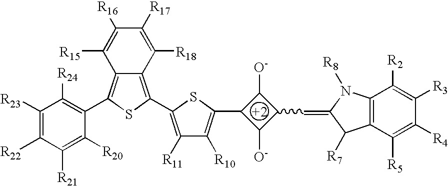

wherein each R₂-R₅, R₇-R₈, R₁₀-R₁₁, R₁₅-R₁₈, and R₂₀-R₂₄, are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

In a preferred embodiment, the compound has the following formula (compound 19, NIAD10):

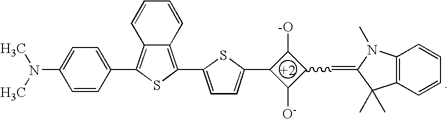

According to a further aspect of the invention, compounds of the following formula are provided:

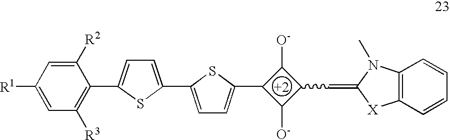

wherein, R¹, R², and R³ are independently —OH, —NMe₂, —N(CH₂CH₂OH)₂, or hydrogen; there is at least one substituent R¹-R³ different from hydrogen; R⁴ is independently Me or —(CH₂)₃—SO₂O—, with the Me group requiring Cl⁻ as a counteranion; and X is —C(Me₂)—, S, O, or NH.

According to a further aspect of the invention, compounds of the following formula are provided:

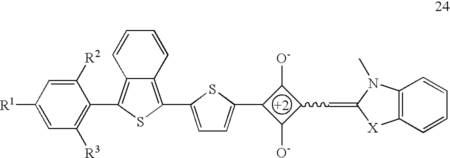

wherein, R¹, R², and R³ are independently —OH, —NMe₂, —N(CH₂CH₂OH)₂, or hydrogen; there is at least one substituent R¹-R³ different from hydrogen; R⁴ is independently Me or —(CH₂)₃—SO₂O—, with the Me group requiring Cl⁻ as a counteranion; and X is —C(Me₂)—, S, O, or NH.

According to a further aspect of the invention, compounds of the following formula are provided:

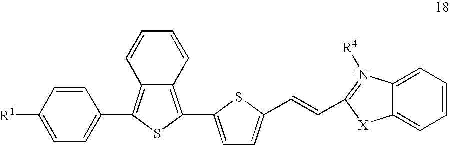

wherein R¹ is independently —OH, —NMe₂, —N(CH₂CH₂OH)₂, or hydrogen; R⁴ is independently Me or —(CH₂)₃—SO₂O—, with the Me group requiring Cl⁻ as a counteraction; and X is —C(Me₂)—, S, O, or NH.

According to a further aspect of the invention, compounds of the following formula are provided:

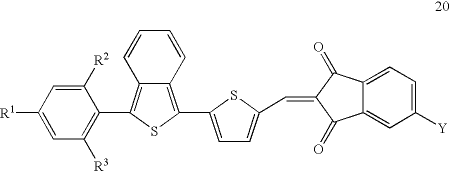

wherein R¹, R², and R³ are independently —OH, —NMe₂, —N(CH₂CH₂OH)₂, or hydrogen; there is at least one substituent R¹-R³ different from hydrogen; and Y is either hydrogen or —COOH.

According to a further aspect of the invention, compounds of the following formula are provided:

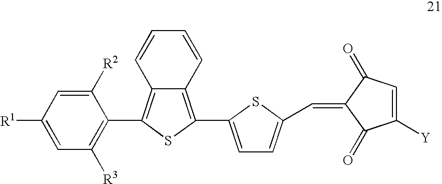

wherein, R¹, R², and R³ are independently —OH, —NMe₂, —N(CH₂CH₂OH)₂, or hydrogen; there is at least one substituent R¹-R³ different from hydrogen; and Y is either hydrogen or —COOH.

In certain embodiments, the foregoing compounds can be modified such that at least one of the substituents R₁-R₂₇ is: ¹³¹I, ¹²³I, ⁷⁶Br, ⁷⁵Br, ¹⁸F, CH₂—CH₂—X*, O—CH₂—CH₂—X*, CH₂—CH₂—CH₂—X*, or O—CH₂—CH₂—CH₂—X* wherein X*-¹³¹I, ¹²³I, ⁷⁶Br, ⁷⁵Br or ¹⁸F, ¹⁹F, or ¹²⁵I. In additional embodiments, a carbon-containing substituent in the foregoing compounds can contain at least one carbon that is ¹¹C, ¹³C or ¹⁴C.

In preferred embodiments, the foregoing compounds bind to amyloid with a dissociation constant (KD) between 0.0001 μM and 10.0 μM when measured by binding to synthetic amyloid peptide or Alzheimer's disease brain tissue.

According to another aspect of the invention, pharmaceutical preparations for in vivo imaging of amyloid deposits are provided. The preparations include any of the foregoing compounds and a pharmaceutically acceptable carrier.

In another aspect of the invention, methods for synthesizing the foregoing compounds in which at least one of the substituents R₁-R₂₇: ¹³¹I, ¹²⁵I, ¹²³I, ⁷⁶Br, ⁷⁵Br, ¹⁸F, or ¹⁹F are provided. The methods include labeling a compound wherein at least one of the substituents is a tri-alkyl tin, by reaction of the compound with a ¹³¹I, ¹²⁵I, ¹²³I, ⁷⁶Br, ⁷⁵Br, ¹⁸F, or ¹⁹F containing substance.

According to still another aspect of the invention, methods for in vivo imaging of amyloid deposits are provided. The methods include administering a detectable amount of the foregoing compounds or pharmaceutical preparation to a subject suspected of having amyloid deposits, and detecting the compound to image the amyloid deposit. In certain embodiments, the amyloid deposit is located in the brain of a subject. Preferably, a ratio of (i) binding of the compound to a brain area other than the cerebellum to (ii) binding of the compound to the cerebellum, in the subject, is compared to the ratio of (i) to (ii) in normal subjects.

In preferred embodiments, the subject is suspected of having a disease or syndrome that is: Alzheimer's Disease, familial Alzheimer's Disease, Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, inclusion body myositis, multiple sclerosis, or homozygotes for the apolipoprotein E4 allele.

In other embodiments, detecting the compound is carried out using infrared imaging, multiphoton imaging, gamma imaging, magnetic resonance imaging or magnetic resonance spectroscopy. In some preferred embodiments, the method includes infrared imaging. In other preferred embodiments, the method includes gamma imaging; preferably the gamma imaging is either PET or SPECT. In still other preferred embodiments, the method is performed using microscopy.

In further embodiments the pharmaceutical composition is administered by intravenous injection.

According to still another aspect of the invention, the compounds are detectably labeled. Preferred detectable labels include radiolabels, fluorescent labels, enzymes, and chemiluminescent molecules.

The invention provides in another aspect methods of evaluating a treatment for an amyloid-associated disorder. The methods include administering a first detectable amount of one or more of the foregoing compounds to a subject undergoing treatment for an amyloid-associated disorder to obtain a first level of binding of the compound(s) to amyloid in the subject, detecting the compound(s) bound to amyloid to determine the first level of binding of the compound(s), administering a second detectable amount of the compound(s), wherein the second administration is at a time subsequent to the first administration, to obtain a second level of binding of the compound(s) to amyloid in the subject, detecting the compound(s) bound to amyloid to determine the second level of binding of the compound(s), and comparing the first level of binding with the second level of binding as an indication of the effectiveness of the treatment on the level of amyloid in the subject.

According to a further aspect of the invention, methods of selecting a treatment for an amyloid-associated disorder in a subject are provided. The methods include administering a detectable amount of one or more of the foregoing compounds to a subject, to obtain a level of binding of the compound to amyloid, detecting the compound(s) bound to amyloid to determine the level of binding of the compound(s), and selecting the treatment for the amyloid-associated disorder based at least in part on the level of binding obtained.

According to another aspect of the invention, methods for determining regression, progression or onset of an amyloid-associated disorder are provided. The methods include administering a detectable amount of one or more of the foregoing compounds to a subject to obtain a level of binding of the compound(s) to amyloid, detecting the compound(s) bound to amyloid to determine the level of binding of the compound(s), and comparing the level of binding of the compound(s) to a control level of binding of the compound(s) as a indication of regression, progression or onset of the condition.

In the foregoing aspects it is preferred that the amyloid-associated disorder is Alzheimer's Disease, familial Alzheimer's Disease, Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, inclusion body myositis, multiple sclerosis, or homozygotes for the apolipoprotein E4 allele.

Similar diagnostic methods are also provided in which the foregoing compounds are conjugated to a binding molecule that selectively binds to a molecule of interest. Exemplary binding molecules include antibodies and binding fragments thereof.

These and other aspects of the invention will be understood with reference to the drawings and the detailed description below.

### BRIEF DESCRIPTION OF THE DRAWINGS

FIG. 1 shows the absorption and emission spectra in methanol solution for compound NIAD6.

FIG. 2 shows the absorption spectrum in methanol solution for compound NIAD7.

FIG. 3 shows the absorption and emission spectra in tetrahydrofuran (THF) solution for compound NIAD8.

FIG. 4 shows the absorption and emission spectra in dimethyl sulfoxide (DMSO) solution for compound NIAD9.

FIG. 5 shows the absorption and emission spectra in DMSO solution for compound NIAD10.

### DETAILED DESCRIPTION OF THE INVENTION

The present invention exploits the ability of several dye compounds and radiolabeled derivatives thereof to cross the blood brain barrier in vivo and bind to Aβ deposited in neuritic (but not diffuse) plaques, to Aβ deposited in cerebrovascular amyloid, and to the amyloid consisting of the protein deposited in neurofibrillary tangles.

The compounds of the present invention have each of the following characteristics: (1) specific binding to synthetic Aβ in vitro and (2) ability to cross a non-compromised blood brain barrier in vivo.

The core structures of some detectable compounds of the invention are based on the general compound structures presented in Table 1, and are referred to herein as compounds 1-9, and 20. Specific detectable compounds of the invention include, in part, the specific compounds described in Table 2, and are referred to herein as compounds 10-19 and 21-30. Each of compounds 10-19 and 21-30 is based on one of the general structures presented in Table 1, and has one or more specific substituents as described herein and in Table 1. The compounds of the invention also include compounds that have additional and/or substituted substituents as described herein. Additional examples of compounds and structures are provided in the Examples section.

TABLE 1
Generic Detectable Compound Structures

| | |
|---|---|
| Compound 1 | 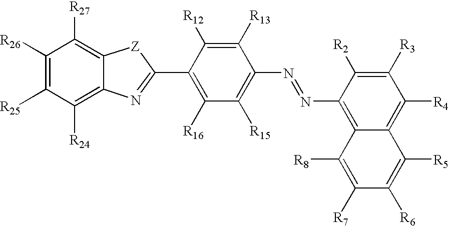 |
| Compound 2 | 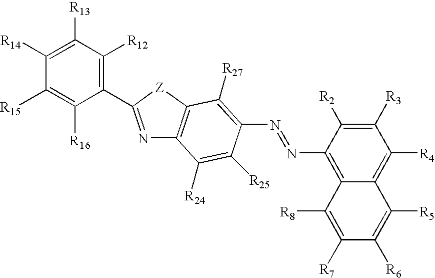 |
| Compound 3 | 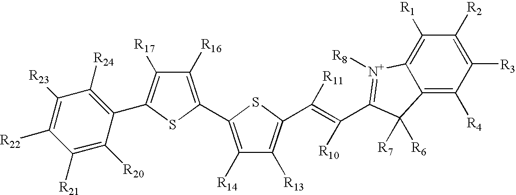 |
| Compound 4 | 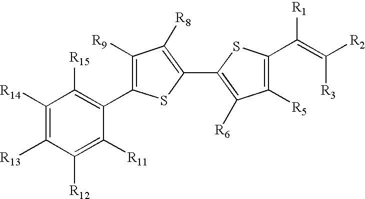 |
| Compound 5 | 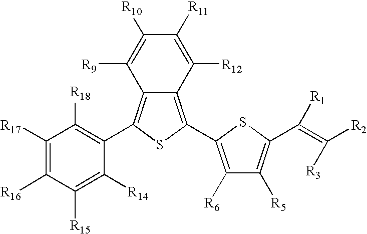 |
| Compound 6 | 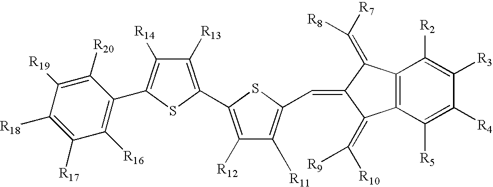 |
| Compound 7 | 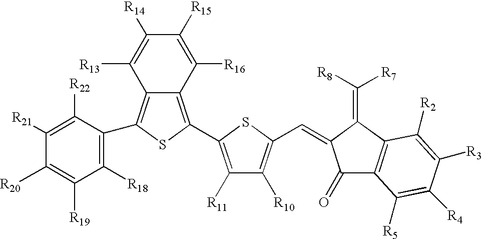 |
| Compound 8 | 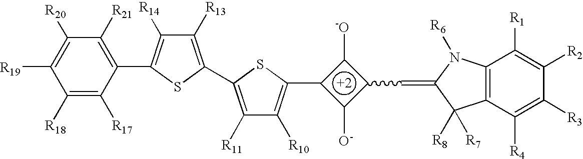 |
| Compound 9 | 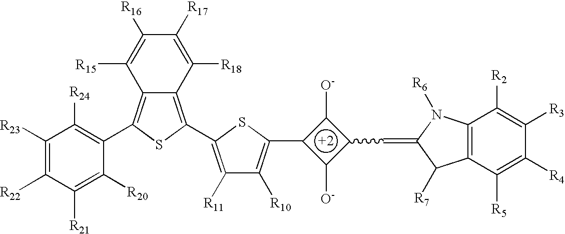 |
| Compound 20 | 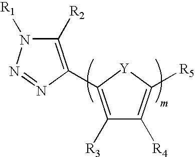 |

where m can be 1, 2, or 3 and R₅ is H or an electron withdrawing group.

TABLE 2
Specific detectable compound examples

| | |
|---|---|
| RD1, Compound 10 | 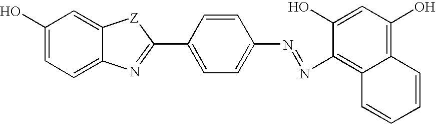 |
| RD2, Compound 11 | 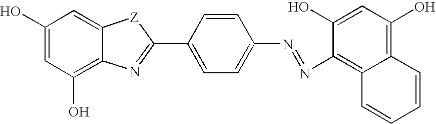 |
| NIAD1, Compound 12 | 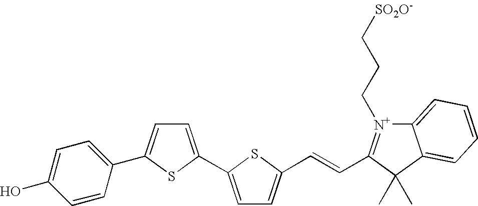 |
| NIAD3, Compound 13 | 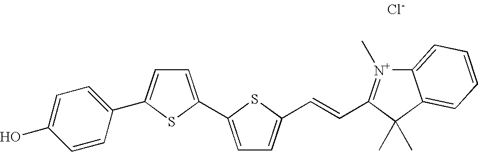 |
| NIAD4, Compound 14 | 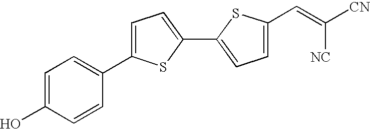 |
| NIAD6, Compound 15 | 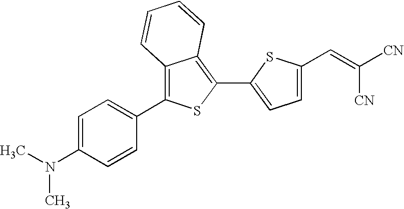 |
| NIAD7, Compound 16 | 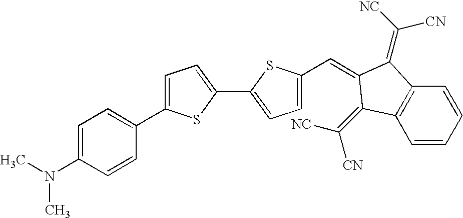 |
| NIAD8, Compound 17 | 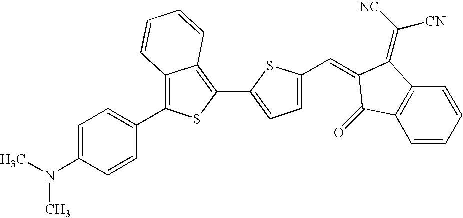 |
| NIAD9, Compound 18 | 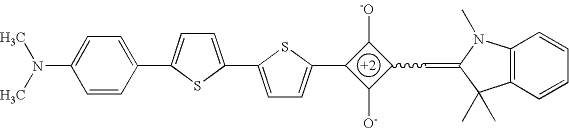 |
| NIAD10, Compound 19 | 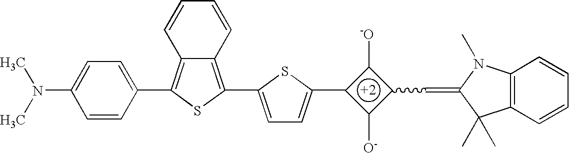 |
| Compound 21 | 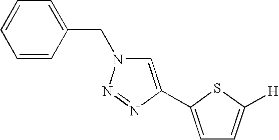 |
| Compound 22 | 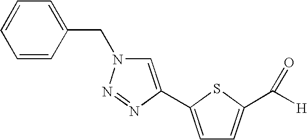 |
| NIAD18, Compound 23 | 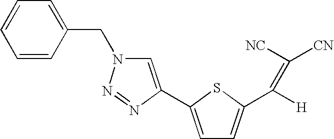 |
| Compound 24 | 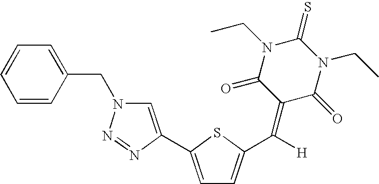 |
| Compound 25 | 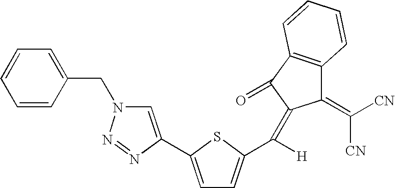 |
| NIAD17, Compound 26 | 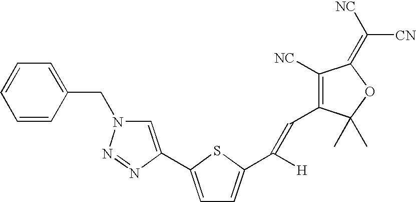 |
| Compound 27 | 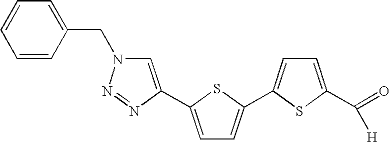 |
| Compound 28 | 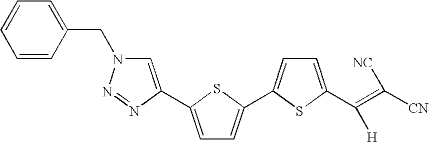 |
| NIAD19 (Compound 29) | 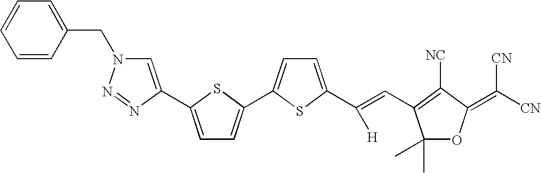 |

The methods of the invention, in part, include the determination of the presence and location of amyloid deposits in an organ or body area, preferably brain, spinal cord, and/or blood vessels of a patient. Certain of the methods of the invention include administration of a detectable quantity of a pharmaceutical composition containing an amyloid-binding compound described herein and analogues thereof, also referred to as a “detectable compound”, or a pharmaceutically acceptable water-soluble salt thereof, to a patient. In some embodiments of the invention, a detectable compound is a radioactively labeled compound, and in some embodiments of the invention a detectable compound is a fluorescent or fluorescently labeled compound. A “detectable quantity” or “detectable amount” means that the amount of the detectable compound that is administered is sufficient to enable detection of the compound bound to amyloid. An “imaging effective quantity” of “imaging effective amount” means that the amount of the detectable compound that is administered is sufficient to enable imaging of binding of the compound to amyloid.

The invention employs detectable compounds which, in conjunction with non-invasive neuroimaging techniques such as magnetic resonance spectroscopy (MRS), imaging (MRI), or gamma imaging such as positron emission tomography (PET), single-photon emission computed tomography (SPECT), or multiphoton imaging may be used to quantify amyloid deposition in vivo. The term “in vivo imaging” refers to any method which permits the detection of a compound in vivo as described herein.

In some embodiments, in vivo imaging includes imaging of compounds with near infrared (NE) spectra. NIR imaging can be non-invasive imaging using NR light and diffuse optical tomography. Methods of NIR imaging are known in the art and examples of methods and compounds for NIR imaging are described in Hintersteiner, M., et al., Nature Biotechnology 2005 May; 23(5):577-83. Epub 2005 Apr. 17. Compounds of the invention useful for NIR imaging are compounds that fluoresce in the NIR region, and are compounds that bind to amyloid-β deposits. In certain embodiments of the invention, gamma imaging may be used to image amyloid-β deposits. Gamma-imaging methods of the invention may include the use of compounds that are modified for radioactive imaging.

In addition to the use of the NM fluorescent compounds of the invention for the assessment of amyloid-β deposits, the NIR compounds of the invention are also useful as NIR fluorophores and may be used to label proteins or other molecules to track a wide range of targets non-invasively. The NIR compounds of the invention and may be used in vitro and/or in vivo to label proteins or other molecules. Thus, the NIR compounds of the invention can be used to determine the presence or absence of and/or to monitor proteins or other molecules in cells and/or tissues.

As used herein, the term “subject” means a mammal, including humans, non-human primates, dogs, cats, horses, pigs, cattle, sheep, and rodents, including but not limited to mice and rats. In some embodiments, the mammal is a human suspected of having, or at risk of having dementia, which may be associated with Alzheimer's disease. In some embodiments of the invention, the subject is suspected of having, or is at risk of having, an amyloid-associated disorder. As used herein, the term “at risk” means having an increased likelihood of having or acquiring a disorder. Factors that can be assessed to determine whether a subject is at risk for an amyloid-associated disorder may include a subject's medical history, age, genetic profile, and gender and may also include the subject's family medical history, genetic profile, etc.

The methods and compositions of the invention are useful in the diagnosis of diseases associated with amyloid β deposition (e.g. amyloid-associated disorders) including, but not limited to: Alzheimer's disease, Down's syndrome, cerebrovascular amyloidosis (Cerebral Amyloid Angiopathy, CAA), Hereditary Amyloidosis with Cerebral Hemorrhage of the Dutch Type (HCHWA-D), Familial British Dementia, vascular dementia, inclusion body myositis, multiple sclerosis, and homozygotes for the apolipoprotein E4 allele, Lewy body disease, and type 2 diabetes mellitus.

As described herein, detectable compounds of the invention are designed to allow fluorescent detection. The detectable compounds of the invention include thiophenes, which are five member heterocycles that contain a ring sulfur. Thiophenes are alternatively known as thiacyclopentadiene; CP 34; furan, thio-; Huile HSO; Huile H50; thiaphene; thiofuram; thiofuran; thiofurfuran; thiole; thiophen; thiotetrole; divinylene sulfide; USAF ek-1860; thiofen; UN 2414; and Hopkin's lactic acid reagent. The detectable compounds of the invention may also include thiophene derivatives.

In some important embodiments of the invention, the detectable compounds include a thiophene or benzothiophene structure. In the thiophene or benzothiophene-containing detectable compounds of the invention, the thiophene or benzothiophene is between (e.g. “bridges”) the region of the compound that is the donor region and the region of the compound that is the acceptor region. As used herein, the donor region comprises an electron donating group and the acceptor region comprises an electron withdrawing group. Acceptor region groups may be H or an electron withdrawing group (EWG). Examples of EWG include, but are not limited to: F, Br, C₁, CF₃, CN, CHO, CONH₂, COOH, COOR′, COCH₃, COR′, NO₂, CON(CH₃)₂, CONR′₂, COOCH₃, COOR′, SO₃H, SO₃R′, CCl₃, NH₄⁺, NR′₃⁺, NR′₄⁺ wherein R′ is a lower alkyl group other than CH₃,

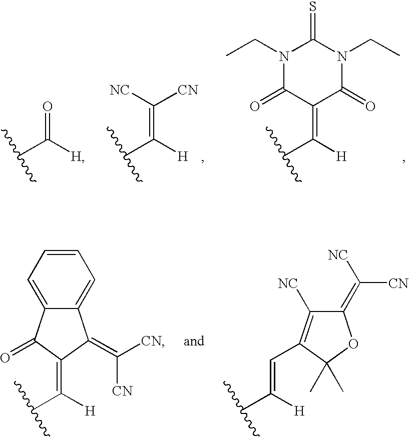

An general example of a detectable compound of the invention that includes a donor region and an acceptor region and a thiophene bridge region is illustrated in Example 1. Additional examples of thiophene and benzothiophene containing compounds and their components are provided in Examples 2-5. The inclusion of the thiophenes results in wave-length shifts and emission modulation that is associated with the binding of the compound to Aβ.

In some embodiments of the invention, a detectable compound of the invention will have decreased fluorescence efficiency in solution and an improved efficiency when bound to amyloid plaques, thus allowing differentiation of bound from unbound detectable compound in a subject, tissue, or sample. In some embodiments, the compounds of the invention may include additional detectable labels, such as radioactive labels, fluorescent labels, etc., as described elsewhere herein.

For purposes of in vivo imaging, the type of detection instrument available is a major factor in selecting a given label and will guide the selection of the radionuclide or stable isotope. For instance, the radionuclide chosen must have a type of decay detectable by a given type of instrument. Radioactive isotopes and ¹⁹F are particularly suitable for in vivo imaging in the methods of the present invention. Suitable radioisotopes for purposes of this invention include beta-emitters, gamma-emitters, positron-emitters, and x-ray emitters. These radioisotopes include ¹³¹I, ¹²³I, ¹⁸F, ¹¹C, ⁷⁵Br, and ⁷⁶Br. Suitable stable isotopes for use in Magnetic Resonance Imaging (MRI) or Magnetic Resonance Spectroscopy (MRS), according to this invention, include ¹⁹F and ¹³C. Suitable radioisotopes for in vitro quantification of amyloid in homogenates of biopsy or post-mortem tissue include ¹²⁵I, ¹⁴C, and ³H. The preferred radiolabels are ¹¹C or ¹⁸F for use in PET in vivo imaging, ¹²³I for use in SPECT imaging, ¹⁹F for MRS/MRI, and ³H or ¹⁴C for in vitro studies. However, any conventional method for visualizing diagnostic probes can be utilized in accordance with this invention.

Another consideration relates to the half-life of the radionuclide. The half-life should be long enough so that it is still detectable at the time of maximum uptake by the target, but short enough so that the host does not sustain deleterious radiation. The radiolabeled compounds of the invention can be detected using gamma imaging wherein emitted gamma irradiation of the appropriate wavelength is detected. Methods of gamma imaging include, but are not limited to, SPECT and PET. Preferably, for SPECT detection, the chosen radiolabel will lack a particulate emission, but will produce a large number of photons in a 140-200 keV range. For PET detection, the radiolabel will be a positron-emitting radionuclide such as ¹⁹F which will annihilate to form two 511 keV gamma rays which will be detected by the PET camera. Methods for multiphoton fluorescence excitation of a compound such as PIB include, but are not limited to, use of a 750-nm light from a mode-locked Ti:Sapphire laser, with fluorescence emission collected using a photomultiplier tube and an interference filter centered at 440 nm.

In the present invention, detectable compounds are made which are useful for in vivo imaging and quantification of amyloid deposition. The detectable compounds described herein and analogues thereof are to be used in conjunction with non-invasive neuroimaging techniques such as magnetic resonance spectroscopy (MRS) or imaging (MRI), positron emission tomography (PET), single-photon emission computed tomography (SPECT), and multiphoton imaging. In accordance with this invention, the compounds described herein and analogues thereof may be labeled with ¹⁹F or ¹³C for MRS/MRI by general organic chemistry techniques known to the art. See, e.g., March, J. ADVANCED ORGANIC CHEMISTRY: REACTIONS, MECHANISMS, AND STRUCTURE (3rd Edition, 1985), the contents of which are hereby incorporated by reference. The compounds described herein and analogues thereof also may be radiolabeled with ¹⁸ F, ¹¹C, ⁷⁵Br, or ⁷⁶Br for PET by techniques that are well known in the art and are described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, NY 1986) the contents of which are hereby incorporated by reference. The compounds described herein and analogues thereof also may be radiolabeled with ¹²³I for SPECT by any of several techniques known to the art. See, e.g., Kulkami, Int. J. Rad. Appl. & Inst. (Part B) 18: 647 (1991), the contents of which are hereby incorporated by reference. In addition, the compounds described herein and analogues thereof may be labeled with any suitable radioactive iodine isotope, such as, but not limited to ¹³¹I, ¹²⁵I, or ¹²³I, by iodination of a diazotized amino derivative directly via a diazonium iodide, see Greenbaum, F. Am. J. Pharm. 108: 17 (1936), or by conversion of the unstable diazotized amine to the stable triazene, or by conversion of a non-radioactive halogenated precursor to a stable tri-alkyl tin derivative which then can be converted to the iodo compound by several methods well known to the art. See, Satyamurthy and Barrio J. Org. Chem. 48: 4394 (1983), Goodman et al., J. Org. Chem. 49: 2322 (1984), and Mathis et al., J. Labell. Comp. and Radiopharm. 1994: 905; Chumpradit et al., J. Med. Chem. 34: 877 (1991); Zhuang et al., J. Med. Chem. 37: 1406 (1994); Chumpradit et al., J. Med. Chem. 37: 4245 (1994). For example, a stable triazene or tri-alkyl tin derivative of the compounds described herein is reacted with a halogenating agent containing ¹³¹I, ¹²⁵I, ¹²³I, ⁷⁶Br, ⁷⁵Br, ¹⁸F or ¹⁹F. Thus, the stable tri-alkyl tin derivatives of the compounds described herein and analogues thereof are novel precursors useful for the synthesis of many of the radiolabeled compounds within the present invention. As such, these tri-alkyl tin derivatives are embodiments of this invention.

The compounds described herein and analogues thereof also may be radiolabeled with known metal radiolabels, such as Technetium-99m (^{99m}Tc). Modification of the substituents to introduce ligands that bind such metal ions can be effected without undue experimentation by one of ordinary skill in the radiolabeling art. The metal radiolabeled thioflavin derivative can then be used to detect amyloid deposits. Preparing radiolabeled derivatives of ^{99m}Tc is well known in the art. See, for example, Zhuang et al., “Neutral and stereospecific Tc-99m complexes: [99 mTc]N-benzyl-3,4-di-(N²-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2):135-40, (1998); and Hom et al., “Technetium-99m-labeled receptor-specific small-molecule radiopharmaceuticals: recent developments and encouraging results” Nuclear Medicine & Biology 24(6):485-98, (1997).

The methods of the present invention may use isotopes detectable by nuclear magnetic resonance spectroscopy for purposes of in vivo imaging and spectroscopy. Elements particularly useful in magnetic resonance spectroscopy include ¹⁹F and ¹³C.

As described above, the compounds of the invention also include compounds that have additional and/or substitutions of one or more of their substituents. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds. For purposes of this invention, heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention. In certain embodiments, the present invention relates to a compound represented by any of the structures outlined herein, wherein the compound is a single stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof (e.g., functioning as targeting Aβ, wherein one or more simple variations of substituents are made which do not adversely affect the targeting activity of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants, which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

Examples of some substitutions to the basic structure of compounds of the invention include, but are not limited to: the following, which may be independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃, and (CH₂)₃SO₂O⁻.

In the compounds and compositions of the invention, the term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In preferred embodiments, a straight chain or branched chain alkyl has 12 or fewer carbon atoms in its backbone (e.g., C₁-C₁₂ for straight chain, C₃-C₁₂ for branched chain), and more preferably 6 or fewer, and even more preferably 4 or fewer. Likewise, preferred cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure, and even more preferably from one to four carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

As used herein, the term “halogen” designates —F, —Cl, —Br or —I; the term “sulfhydryl” means —SH; and the term “hydroxyl” means —OH.

The term “methyl” refers to the monovalent radical —CH₃, and the term “methoxyl” refers to the monovalent radical —CH₂OH.

The term “aralkyl” or “arylalkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, —C(O)NHOH, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms “ortho”, “meta” and “para” apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” or “heteroaryl” refer to 3- to 10-membered ring structures, more preferably 3- to 7-membered rings, whose ring structures include one to four heteroatoms. Heterocycles can also be polycycles. Heterocyclyl groups include, for example, thiophene, benzothiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, —C(O)NHOH, silyl, ether, alkylthio, sulfonyl, ketone, aldehyde, ester, a heterocyclyl, an aromatic or heteroaromatic moiety, —CF₃, —CN, or the like.

As used herein, the definition of each expression, e.g. alkyl, m, n, etc., when it occurs more than once in any structure, is intended to be independent of its definition elsewhere in the same structure.

The compounds and methods of the invention may be used to diagnose Alzheimer's disease, including the diagnosis of early-stage though advanced-stage AD. In addition, the methods provided may be used for the diagnosis of clinically confusing cases of dementia and may be used to rule in or to rule out Alzheimer's disease as a diagnosis in a subject. The methods of the invention may also be used in the diagnosis of other amyloid-associated disorders. Amyloid-associated disorders are disorders in which the deposition of amyloid-β is characteristic. As used herein, amyloid-associated disorders include diseases such as Alzheimer's disease, Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, inclusion body myositis, multiple sclerosis, and bomozygotes for the apolipoprotein E4 allele.

The methods of the invention are useful for longitudinal studies of amyloid deposition in human populations at risk for amyloid deposition, e.g. a subject suspected of having or at risk for having an amyloid-associated disorder. The methods of the invention permit the level of amyloid deposition to be followed over time, allowing the determination of the correspondence between the timing of the deposition of amyloid-β relative to the onset of clinical symptoms. Thus, the methods of the invention can be utilized to determine whether the level and timing of amyloid-β deposition corresponds to amyloid-associated disease symptoms and severity. The methods of the invention can also be used to monitor the effectiveness of therapies targeted at preventing amyloid deposition. For example, a baseline level of amyloid deposition can be obtained in a subject, a subsequent determination of the level of amyloid deposition can be done, and the two levels compared. Such a comparison can provide information from the subject over time, allowing the assessment of efficacy of treatments provided to the subject.

The methods of the invention include in part, measuring levels of amyloid B. Levels of amyloid β can be determined in a number of ways when carrying out the various methods of the invention. In one particularly important method, the level of amyloid β is measured by assessing a relative level of binding as described above. Such a relative measure can be expressed, for example, as a percentage of total detectable compound introduced into the subject. For example, in gamma imaging, the relative measure can be expressed as a percentage of the total radiation administered to the subject. Another measurement of the level of amyloid β is a measurement of absolute levels of amyloid β. Another measurement of the level of amyloid β is a measurement of the change in the level of amyloid β over time. This may be expressed in an absolute amount or may be expressed in terms of a percentage increase or decrease over time.

Importantly, levels of amyloid β are advantageously compared to controls according to the invention. The control may be a predetermined value, which can take a variety of forms. It can be a single cut-off value, such as a median or mean. It can be established based upon comparative groups, such as in groups without dementia or indication of risk for dementia and groups having dementia or having an indication of a risk or high risk of dementia. Another example of comparative groups would be groups having a particular disease (e.g. Alzheimer's disease, Down's syndrome, etc), condition or symptoms and groups without the disease, condition or symptoms. Another comparative group would be a group with a family history of a condition (e.g. Alzheimer's disease, Down's syndrome, etc.) and a group without such a family history. The predetermined value can be arranged, for example, where a tested population is divided equally (or unequally) into groups, such as a low-risk group, a medium-risk group and a high-risk group or into quandrants or quintiles, the lowest quandrant or quintile being individuals with the lowest risk or amounts of amyloid-β deposition and the highest quandrant or quintile being individuals with the highest risk or amounts of amyloid-β deposition.

The predetermined value, of course, will depend upon the particular population selected. For example, an apparently healthy population will have a different ‘normal’ range than will a population which is known to have a condition related to abnormal amyloid-β deposition. Accordingly, the predetermined value selected may take into account the category in which an individual falls. Appropriate ranges and categories can be selected with no more than routine experimentation by those of ordinary skill in the art. By abnormally high it is meant high relative to a selected control. Typically the control will be based on apparently healthy normal individuals in an appropriate age bracket. It will also be understood that the controls according to the invention may be, in addition to predetermined values, samples of materials tested in parallel with the experimental materials. Examples include samples from control populations or control samples generated through manufacture to be tested in parallel with the experimental samples.

As mentioned above, it is also possible to characterize amyloid-β levels by monitoring changes in the absolute or relative amounts of amyloid β over time. For example, it is expected that an increase in amyloid β correlates with increasing severity of an amyloid-associated disorder, e.g. correlates with the advancing stages of the disorder. Accordingly one can monitor amyloid-β levels over time to determine if amyloid-β levels of a subject are changing. An increase in the relative or absolute level of amyloid β that is greater than 0.1% may indicate the onset or progression of an amyloid-associated disorder. Preferably, the change in amyloid-β levels, which indicates onset or progression of an amyloid-associated disorder, is greater than 0.2%, greater than 0.5%, greater than 1.0%, 2.0%, 3.0%, 4.0%, 5.0%, 7.0%, 10%, 15%, 20%, 25%, 30%, 40%, 50%, or more. Reductions in amounts of amyloid-β over time may indicate regression of an amyloid-associated condition. Additionally, the absence of significant change in the amount of amyloid β in a subject over time may mean the progression of an amyloid-associated disease has stopped or significantly slowed.

The invention in another aspect provides a diagnostic method to determine the stage of an amyloid-associated disorder. The invention also provides a method that can be used to determine the effectiveness of treatments for amyloid-associated disorders and/or treatments to reduce amyloid-β levels, or to stop an increase in amyloid-β levels. The “evaluation of treatment” as used herein, means the comparison of a subject's levels of amyloid β measured at different measuring times, preferably at least one week apart. The preferred time to obtain the second or subsequence level measurement from the subject is at least one week after obtaining the first measurement, which means the second measurement is obtained at any time following the week of the first measurement, preferably at least 1, 2, 3, 4, 5, 6, 7, 8, 9, 10, 15, 20, 25, 30, 35, 40, 45, 50, 55, 60, 65, 70, 80, 85, 90, 95, 100 or more weeks after the time of first level measurement in the subject.

The comparison of levels of amyloid if in two or more measurements, taken on different days, is a measure of the onset, progression, or regression of an amyloid-associated disorder in a subject, thus provided a method of diagnosis of the amyloid-associated disorder in a subject, The comparison of two or more measurements of the level of amyloid β in a subject allows evaluation of the treatment of the amyloid-associated disorder that has been administered to the subject. For example, an initial measurement of a subject's level of amyloid β may indicate that the subject has an amyloid-associated disorder and based on this assessment, treatment may be initiated in the subject. A subsequent measure of the level of amyloid β from the subject may be used to determine the efficacy of the patient's treatment. Thus, a subsequent measure from a patient may allow the adjustment of therapy for an amyloid-associated disease in a subject. The results of two or more determinations of a subject's amyloid β levels may also be used in conjunction with behavioral measures, e.g. for dementia, and may provide information on the correlation between amyloid β levels and dementia or other clinical manifestations of an amyloid-associated disorder.

As will be appreciated by those of ordinary skill in the art, the evaluation of the treatment also may be based upon an evaluation of the symptoms or clinical end-points of the associated disease, such as the level of dementia and/or the progression of physical and/or mental functions that are characteristic of an amyloid-associated disorder. Thus, the methods of the invention also provide for determining the regression, progression or onset of a condition which is characterized by abnormal levels of amyloid-β deposition. In some instances, the subjects to which the methods of the invention are applied are already diagnosed as having a particular amyloid-associated disorder. In other instances, the measurement will represent the diagnosis of the amyloid-associated disorder. In some instances, the subjects will already be undergoing drug therapy for preventing and or treating an amyloid-associated disorder, while in other instances the subjects will be without present drug therapy for preventing and/or treating an amyloid-associated disorder.

In some instances, the absence of change in the amount of amyloid 0 in subsequent measurements from a subject may indicate that the progression of the amyloid-associated disorder has halted or significantly slowed. The slowing or stopping of the progression of an amyloid-associated disorder in a subject undergoing treatment for an amyloid-associated disorder may be an indicator of the efficacy of the therapy and may be useful to determine and monitor the effective amount of a therapeutic compound for an amyloid-associated disorder.

The detectable compounds disclosed herein have additional utility based on their properties as near infrared (NIR) fluorophores. Compared to existing NIR fluorophores, the compounds described herein are very small and thus can be used to label proteins or other molecules to track a range of molecules non-invasively. Accordingly, similar to the methods described herein for measurement of amyloid β and diagnostic methods for amyloid-associated disorders, and based on their advantageous near infrared spectral properties, the compounds described herein can be used in additional diagnostic methods. The compounds can be coupled to antibodies or other molecules that bind selectively to a cellular molecule of interest. In such embodiments, the binding molecule portion of the conjugate provides the requisite specificity of binding, and the NIR fluorophore molecule provides the detectability. For example, a monoclonal antibody can be coupled to one or more NIR fluorophore molecules of the compounds of the invention for use in diagnostic applications. After administering the antibody-NIR fluorophore conjugate to a subject, the antibody binds to the molecules of interest in the subject, after which the fluorophore is detected to aid in a diagnostic method based on amounts of the molecule of interest.

Any coupling method that does not destroy the binding properties of the binding molecules or the NIR fluorescence properties of the compounds of the invention can be utilized to prepare the conjugates.

The binding molecules can be any molecule that has suitably selective binding properties, including specificity of binding (e.g., low cross-reactivity) and avidity of binding. These properties and suitable molecules are known to one of ordinary skill in the art or can be identified by routine experimentation. In certain embodiments, the binding molecules are antibodies or binding fragments thereof.

Antibodies may be produced using standard techniques well known to the art. Standard reference works setting forth the general principles of antibody production include Catty, D., Antibodies, A Practical Approach, Vol. 1, IRL Press, Washington D.C. (1988); Klein, J., Immunology: The Science of Cell-Non-Cell Discrimination, John Wiley and Sons, New York (1982); Kennett, R., et al., Monoclonal Antibodies Hybridoma, A New Dimension In Biological Analyses, Plenum Press, New York (1980); Campbell, A., Monoclonal Antibody Technology, in Laboratory Techniques and Biochemistry and Molecular Biology, Vol. 13 (Burdon, R. et al. EDS.), Elsevier Amsterdam (1984); and Eisen, H. N., Microbiology, third edition, Davis, B. D. et al. EDS. (Harper & Rowe, Philadelphia (1980).

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “chimeric” and “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762 and 5,859,205.

Fully human monoclonal antibodies also can be prepared by immunizing mice transgenic for large portions of human immunoglobulin heavy and light chain loci. See, e.g., U.S. Pat. Nos. 5,545,806, 6,150,584, and references cited therein. Following immunization of these mice (e.g., XenoMouse (Abgenix), HuMAb mice (Medarex/GenPharm)), monoclonal antibodies can be prepared according to standard hybridoma technology. These monoclonal antibodies will have human imunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies.

The antibodies of the present invention thus are prepared by any of a variety of methods, including administering a molecule of interest, fragments of the molecule of interest, cells expressing the molecule of interest or fragments thereof, and the like to an animal to induce polyclonal antibodies. The production of monoclonal antibodies is according to techniques well known in the art.

In some embodiments of the invention, after a sufficient time has elapsed following administration for the detectable compound to bind with the amyloid, for example 30 minutes to 48 hours, the area of the subject under investigation is examined by routine imaging techniques such as MRS/MRI, SPECT, planar scintillation imaging, PET, and any appropriate imaging methods known to those of skill in the art.

Protocols for administration and determining the level of a detectable compound of the invention will necessarily vary depending upon factors specific to the patient, as noted above, and depending upon the body site under examination, method of administration and type of label used; the determination of specific procedures would be routine to the skilled artisan. For example with gamma imaging, the radiation emitted from the organ or area being examined can be measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound.

In some embodiments an internal control may be used to determine a relative amount of binding of an amyloid-binding detectable compound of the invention. In some embodiments, the total level of detectable compound in the tissue or region of interest in a test subject can be compared to the total level of detectable compound in the same region of a control subject. In some instances, the control level may be the level previously obtained from the same region of the same test subject. In some embodiments of the invention, level of detectable compound in the region of interest in a subject (region 1) can be determined along with the level of the detectable compound in another (control) region (region 2) of the subject's body. In some instances, for example in a subject suspected of having or at risk of having Alzheimer's disease, the region of interest will be the cerebellum. The ratio of the level of detectable compound in region 1 to the detectable compound in region 2 can be compared to ratio of measurements taken from regions 1 and 2 of a normal control subject. Thus, in brain imaging, the amount (total or specific binding) of the bound detectable compound of the invention may be measured and compared (as a ratio) with the amount of detectable compound of the invention bound to the cerebellum of the subject. This ratio is then compared to the same ratio in age-matched normal brain, which serves as a control.

In some embodiments, the amyloid β in a tissue or region or interest is be measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound.

The invention also includes methods with which amyloid deposition in may be identified, and/or measured in biopsy or post-mortem tissue. Thus, some embodiments of the invention include incubating formalin-fixed tissue with a solution of a detectable compound of the invention, for example one of compounds 10-19 as provided herein. Preferably, the solution is 25-100% ethanol, (with the remainder being water) saturated with a detectable compound of the invention. Upon incubation, the detectable compound binds to and/or labels the amyloid deposit in the tissue, allowing detection (e.g. visualization) of the amyloid deposit by any standard method. Detection methods useful in this aspect of the invention may include microscopic techniques such as bright-field, fluorescence, laser-confocal and cross-polarization microscopy.

The method of quantifying the amount of amyloid in biopsy or post-mortem tissue involves incubating a detectable compound of the invention, or a water-soluble, non-toxic salt thereof, with homogenate of biopsy or post-mortem tissue. The tissue is obtained and homogenized by methods well known in the art. The detectable compound may include a radiolabel or fluorescent label or other detectable label such as enzymes, chemiluminescent molecules, etc, which are well known to skilled artisans. In some embodiments, the radiolabel is ¹²⁵I, ¹⁴C or ³H which is contained in a substituent substituted on one of the compounds of the invention. Tissue containing amyloid deposits will bind to the detectable compound of the invention and the bound tissue is then separated from the unbound tissue by any mechanism known to the skilled artisan, such as filtering. The bound tissue can then be quantified through any means known to the skilled artisan (e.g. scintillation counting, densitometry, etc). In some embodiments, the units of tissue-bound detectable label are converted to units of micrograms of amyloid per mg of tissue by comparison to a control. An example of a control useful in the methods of the invention is a standard curve generated by incubating known amounts of amyloid with the detectable compound of the invention.

The method of distinguishing an Alzheimer's diseased brain from a normal brain involves obtaining tissue from (i) the cerebellum and (ii) another area of the same brain, other than the cerebellum, from normal subjects and from subjects suspected of having Alzheimer's disease. Such tissues are made into separate homogenates using methods well known to the skilled artisan, and then are incubated with a detectable compound of the invention. The amount of tissue which binds to the detectable compound of the invention is then calculated for each tissue type (e.g. cerebellum, non-cerebellum, normal, abnormal) and the ratio for the binding of non-cerebellum to cerebellum tissue is calculated for tissue from normal and for tissue from patients suspected of having Alzheimer's disease. These ratios are then compared. For example, if the ratio from the brain suspected of having Alzheimer's disease is above about 90% of the ratios obtained from normal brains, the diagnosis of Alzheimer's disease is made. The normal ratios can be obtained from previously obtained data, or alternatively, can be recalculated at the same time the suspected brain tissue is studied. It will be understood that the percentage cut off for diagnosis of Alzheimer's disease may vary depending on the type of detectable label/reporter used. In some embodiments, a ratio that is diagnostic may be up to about 50%, 55%, 60%, 65%, 70%, 75%, 80%, 85%, 95% or more.

It will be understood that the methods of the invention can also be used to obtain an absolute or relative level of binding of a detectable compound of the invention in a tissue of interest and to compare that level to a control level of binding in a control tissue and/or control subject or population for the diagnosis of other amyloid-associated disorders.

The ability of the detectable compounds of the invention to preferentially bind to amyloid plaques rather than neurofibrillary tangles is particularly true at concentrations less than 10 nM, which includes the in vivo concentration range of PET radiotracers. At these low concentrations, significant binding does not result when compared to control brain tissue containing neither plaques nor tangles. However, incubation of homogenates of brain tissue which contains mainly plaques and some tangles with a detectable compound of the invention, results in a significant increase in binding when compared to control tissue without plaques or tangles. This data suggests the advantage that these compounds are specific for Aβ deposits at concentrations less than 10 nM. These low concentrations are then detectable in PET studies, making PET detection using detectable compounds of the invention which are specific for amyloid β deposits possible. The use of such compounds permits PET detection in amyloid β deposits such as those found in plaques and cerebrovascular amyloid. Since it has been reported that amyloid β levels in the frontal cortex are increased prior to tangle formation, this would suggest that detectable compounds of the invention, used as PET tracers, would be specific for the earliest changes in AD cortex. Naslund et al. JAMA 283:1571 (2000).

The pharmaceutical compositions of the present invention include pharmaceutical preparations that, in addition to specifically binding amyloid in vivo and capable of crossing the blood brain barrier, are also non-toxic at appropriate dosage levels and have a satisfactory duration of effect. Accordingly, for therapeutic uses of the compounds of the present invention, a pharmaceutical composition comprising a compound of the invention is administered to subjects who have, or are suspected of having an amyloid-associated disorder.

Thus, according to still another aspect of the invention, methods are provided for treating a subject to reduce the risk of an amyloid-associated disorder. The methods involve selecting and administering to a subject who is known to have an abnormally-high level of amyloid-β deposition, an agent for treating the disorder. In some embodiments, the agent is an agent for reducing amyloid-β levels and is administered in an amount effective to reduce amyloid-β levels. In some embodiments, the agent is an agent for reducing symptoms of the amyloid-associated disorder.

In this aspect of the invention, the treatments are based upon selecting subjects who have elevated levels of amyloid-β disposition. As used herein, the term “elevated” means higher when compared to a control level. Such subjects may already be receiving a drug for prevention or treatment of an amyloid-associated disorder, but, according to the invention, are now candidates for an elevated level of the treatment based upon the presence of the elevated levels of amyloid β. It may be appropriate according to the invention to alter a therapeutic regimen for a subject, based upon the measurement of the level of amyloid β. This can be understood in connection with treatment of amyloid-associated disorders. Subjects who are believed to be at risk of having an amyloid-associated disorder or are known to have an amyloid-associated disorder are treated in at least two different ways. Some subjects perceived to be at risk are treated only with non-drug therapy, such as diet changes and monitoring. Other subjects who are thought likely to have an amyloid-associated condition are treated with oral drug therapy to reduce the progression of the disorder. According to the present invention, as a result of determining an elevated level of amyloid β, an individual undergoing only non-drug therapy may be a candidate for drug therapy as a result of the amyloid-β test. This may result in earlier and more effective treatment of amyloid-associated disorders. In some instances, a subject may be free of any present treatment but may be indicated to be a candidate for a therapy to prevent or treat an amyloid-associated disorder based as a result of the amyloid-β measurement test of the invention. Thus, a subject may be selected and treated for the first time, a subject's treatment may be adjusted to include elevated levels of the same drugs, a subject may be treated with different therapies as a result of the assays of the invention.

According to the present invention, some of the subjects are free of symptoms otherwise calling for treatment with a particular therapy. This means that absent the amyloid-β measurement test, the subject would not according to convention as of the date of the filing of the present application have symptoms calling for treatment with a particular therapy. It is only as a result of the measuring the level of amyloid β with the methods of the invention that the subject becomes a candidate for treatment with the therapy.

Examples of drug therapies (for treatment and/or prophylaxis) that may be administered for the prevention or treatment of Alzheimer's disease include, but are not limited to: trophic factors such as nerve growth factor (NGF), brain-derived neurotrophic factor (BDNF), neurotrophin 3 (NT3), glial cell line-derived neurotrophic factor (GDNF), or ciliary neurotrophic factor (CNTF). Other growth factors that may be delivered to the brain and spinal cord include: neurotrophin 4/5 (NT4/5), leukemia inhibitory factor (LIF), cardiotrophin (CT-1), insulin-like growth factors 1 and 2 (IGF-1, IGF-2), transforming growth factor alpha (TGF-alpha), transforming growth factor beta 1-3 (TGF-beta1, TGF-beta2, TGF-beta3), neurturin (NTN), artemin (ART), persephin (PSP), acidic fibroblast growth factor (FGF-1), basic fibroblast growth factor (FGF-2), fibroblast growth factor-5 (FGF-5), platelet-derived growth factor (PDGF) and stem cell factor (SCF). Other drug therapies (for treatment and/or prophylaxis) of Alzheimer's disease include: include amyloid degrading enzymes for Alzheimer's Disease (e.g., the neprilysin (NEP) family of zinc metalloproteinases, such as NEP and endothelin-converting enzyme, insulysin, angiotensin-converting enzyme, matrix metalloproteinases, plasmin and thimet oligopeptidase (endopeptidase-24.15)); glutamate degrading enzymes; anti-oxidants including SOD1, SOD2, glutathione peroxidase and catalase; anti-apoptotics including Bcl-2, CrmA, baculoviral LAPs and mammalian LAPs (inhibitor of apoptosis proteins including naip, xiap/hilp/miha, c-iapl/hiap-2/mihb, c-iap2/hiap-1/mihc); proteasome enhancers; kinase inhibitors; glutamate transport enhancers (e.g., EAAT2/GLT1); glutamate metabolizers (e.g., glutamate decarboxylase); beta-amyloid protein antibodies; neurotransmitter synthesizing enzymes including GAD, choline acetyl-transferase and tyrosine hydroxylase; compounds that inhibit caspase activity including caspase inhibitors (e.g., Z-Val-Ala-Asp-fluoromethylketone (Z-VAD-fmk); Z-VDVAD-fmk, Z-DEVD-fink, and Z-Asp-cmk (Z-Asp-2,6-dichlorobenzoyl-oxymethylketone)), minocycline and dominant negative caspase mutants; haloperidol; phenothiazines; benzodiazepines; acetylcholine esterase inhibitors (including donepezil, rivastigmine and galantamine); tetrahydroacridinamine (Tacrine); beta- and gamma-secretase inhibitors; Abeta vaccines; Cu—Zn chelators; cholesterol-lowering drugs; non-steroidal anti-inflammatory drugs; carbidopa and/or levodopa with or without a catechol-O-methyl transferase (COMT) inhibitors such as Comtan or Tasmar; dopamine agonists including pramipexole, pergolide, and ropinerol; amantadine; selegiline; gabapentin; lamotrigine; topiramate; vigabatrin; Rilutek® (riluzole); cholinergic agents including pyridostigmine; beta blockers including timolol, levobunolol and betaxolol; parasympathomimetics including pilocarpine, carbachol and phospholine iodide; alpha agonists including apraclonidine, brimonidine and epinephrine; carbonic anhydrase inhibitors including dorzolamide and latanoprost.

Pharmaceutical therapies (for treatment and/or prophylaxis) of other amyloid-associated conditions will be known to those of ordinary skill in the art. The methods of the invention provided herein can be used to monitor the effective amounts, dosing effective conditions, and overall efficacy of these therapies for the prevention and/or treatment of amyloid-associated disorders.

Reducing the risk of a disorder associated with abnormally high levels of amyloid β may include the use of treatments and/or medications to reduce amyloid-β levels, therein reducing, for example, the subject's risk of dementia or vascular complications that may be associated with the amount or level of amyloid-β deposition.

In another aspect, the present invention provides “pharmaceutically acceptable” compositions, which comprise an imaging effective quantity of one or more of the compounds described herein, formulated together with one or more pharmaceutically acceptable carriers (additives) and/or diluents. As described in detail, the pharmaceutical compositions of the present invention may be specially formulated for administration in solid, liquid or aerosolized form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous, intrathecal, intracranial, intraperitioneal, or epidural injection as, for example, a sterile solution or suspension, or sustained-release formulation; topical application, for example, as a crea m, ointment, or a controlled-release patch or spray applied to the skin, lungs, oral cavity, or other mucosal surfaces; intravaginal or intrarectal administration, for example, as a pessary, cream or foam; ocular administration, for example, as a liquid applied to the eye; nasal administration, for example, as a nasal spray; or inhalation into the lungs or nasal cavities, for example, as provided by an inhalation aerosol.

The preparations of the present invention may be given orally, parenterally, topically, rectally, vaginally, or via inhalation into the lungs or nasal cavities. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, transdermal, subdermal, intraperitoneal, transtracheal, subcutaneous, subcuticular, intraarticular, subcapsular, subarachnoid, intraspinal, intracranial and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As set out herein, certain embodiments of the present compounds may contain a basic functional group, such as amino or alkylamino, and are, thus, capable of forming pharmaceutically-acceptable salts with pharmaceutically-acceptable acids. The term “pharmaceutically-acceptable salts” in this respect refers to the relatively non-toxic, inorganic and organic acid addition salts of compounds of the present invention. These salts can be prepared in situ in the administration vehicle or the dosage form manufacturing process, or by separately reacting a purified compound of the invention in its free base form with a suitable organic or inorganic acid, and isolating the salt thus formed during subsequent purification. Representative salts include the hydrobronide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propiolic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, bronchial, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, preferably from about 5% to about 70%, most preferably from about 10% to about 30%.

In certain embodiments, a formulation of the present invention comprises an excipient that may be a cyclodextrin, liposome, micelle forming agent, e.g., bile acids, or polymeric carriers, e.g., polyesters and polyanhydride; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired imaging effect and then gradually increasing the dosage until the desired effect is achieved.

Generally, the dosage of the detectable compounds of the invention will vary depending on considerations such as age, condition, gender, and extent of disease in the patient, contraindications, if any, concomitant therapies and other variables, to be adjusted by a physician skilled in the art. In some embodiments of the invention, dosage can vary from 0.001 μg/kg to 100 mg/kg, preferably 0.005 μg/kg to 100 μg/kg, more preferably 0.01 μg/kg to 1.0 μg/kg. In certain embodiments of the invention, e.g. for NIR fluorescence imaging, dosages will be at the higher end of the foregoing scale and in preferred embodiments are in the range of 0.0 μmg/kg to 10 mg/kg. In some embodiments, the dose administered to a subject may be modified as the physiology of the subject changes due to age, disease progression, weight, or other factor.

### EXAMPLES

### Example 1

We have designed a family of near infrared target dyes with a general structure:

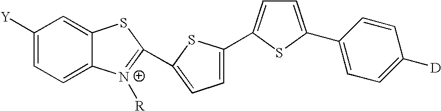

We have designed a target dye with a predicted absorption band edge at 688 mm with Y=Sulfonate, R=Methyl, and D=OH. The thiophenes are elements in the design and the analog without the thiophenes has an absorption band edge at 600 nm.

There are multiple characteristics in addition to the wavelength that are optimized by the choice of the structural variables in this family of far red dyes. The water solubility is balanced with lipophilicity. Y electron withdrawing can have different characteristics. They can be a simple nitrile group (—CN), which is extremely electron withdrawing and uncharged. Alternatively it can be a sulfonate derivative, which can be anionic as in the case of Y═SO₃ or neutral Y═SO₂N(CH₂CH₂OH)₂ or —SO₂NH(CH₂CH₂OH). We further balance the solubility by substituting on of the —CH₂CH₂OH groups with a simple hydrocarbon chain.

The “R” group on the nitrogen can also be varied from simple methyl groups to extended groups with a variety of functionality. In the case that D is a hydroxyl group (—OH) with an acidic proton, the structure can formally become neutral by deprotonation. It is also likely that this acidity alters the emission wavelength and efficiency. The emission is enhanced upon binding to the amyloid plaques. To promote this effect the target dye, by design, has flexible linkages that will result in decreased fluorescence efficiency in solution and an improved efficiency when bound to amyloid plaques.

### Example 2

### Near-Infrared Dyes for Non-Invasive Optical Imaging of Alzheimer Amyloid Plaques in Brain

### 1) Attributes for NIAD

The following properties are incorporated into compounds for application of near-infrared dyes for non-invasive optical imaging of α-amyloid plaques in brain. The spectral properties (absorption and emission wavelength) are in a range of 650-800 nm. Emission occurs with a sufficient fluorescence quantum yield in order to be detectable. The compounds have specific binding to β-amyloid plaques in brain (a desirable binding constant was <100 nM). The emission properties of bound and non-bound dye are substantially different at the same excitation wavelength (e.g., fluorescence quantum yield increases upon binding to β-amyloid aggregates). This condition is involved with enhancement of the contrast of the optical imaging. In addition the compounds have sufficient permeability across the blood-brain barrier.

### 2) Generic Design

The compounds possessing the above mentioned desired attributes are represented by general formula 1. In the formula, D is an aromatic donor group, B—conjugated polarizable bridge, preferably incorporating thiophene or benzo[c]thiophene units, and A—any conjugated acceptor group. Molecular weight of the proposed compounds should be in a range of 300-500 Da, and no more than 700 Da.

D-B-A 1

A feature of the present design of imaging dyes was implementation of thiophene or benzothiophene incorporating bridge. The following structures of the bridge are employed:

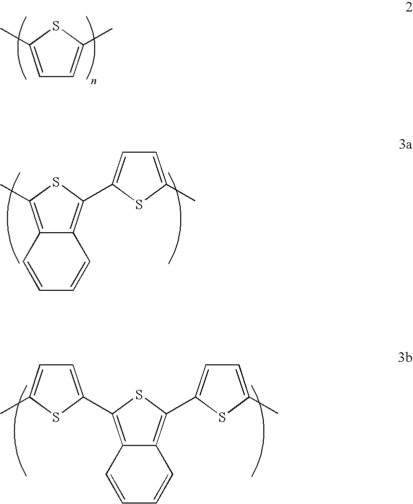

In the structure 2 (above), 11 may be equal to 1, 2, or 3. Utilization of these bridge structures is, thought to be responsible for both the binding selectivity of the proposed dyes and for their useful spectral properties (including change of these properties upon binding to β-amyloid plaques, i.e. imaging contrast enhancement).

As a donor D, hydroxy- or amino-substituted phenyl group is used. As an example, the following structures 4-5 are utilized. In the structure 5, R can be methyl, ethyl, or 2-hydroxyethyl.

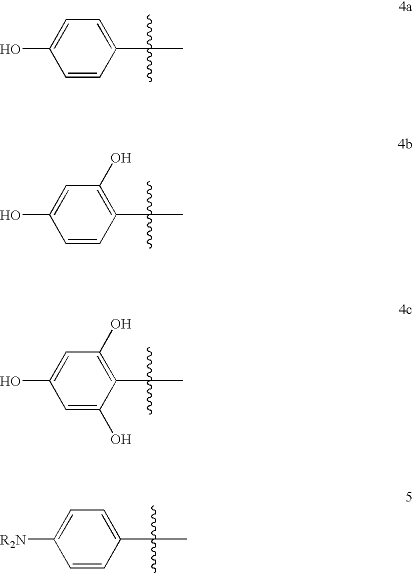

Acceptor A represents differently substituted ethylene group. Possible examples include 2,2-dicyanovinyl 6,1,2,2-tricyanovinyl 7, various derivatives of 8, indan-1,3-dione based groups 9a-b, and related to it sulfone group 10, cyclopenten-1,3-dione moiety 11, or pyridinium group 12. X in 8 may be —C(Me₂)—, O, S, or NH, and substituent R, independently on X, can be either 3-sulfobutyl or methyl group, with the latter substituent requiring counteranion Cl⁻ In the fragments 9, 10, and 11, Y can stand for hydrogen or —COOH, and, independently on Y, Z may be either ═O or ═C(CN)₂.

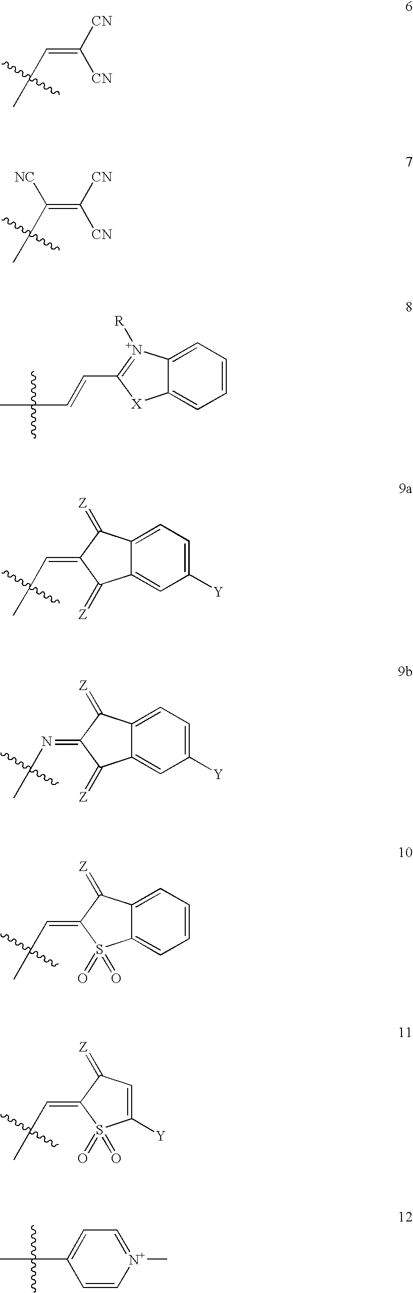

As a special case of the general design 1, unsymmetric squarilium and croconium dyes utilizing “bivalent” acceptor group A are proposed. They can be represented by a general formula 1a:

D-B-A-D′ 1a

Here A is represented by the fragment 12 or 13, with D and B being the same as described above. The second electron-donating group D′ may include, among others, heterocyclic structures of type 15, where X and R are the same as in the fragment 8.

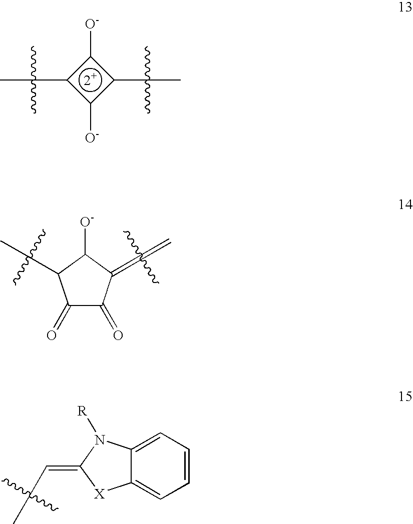

Preferred are compounds of general formula 1, which can be represented by structures 16-22:

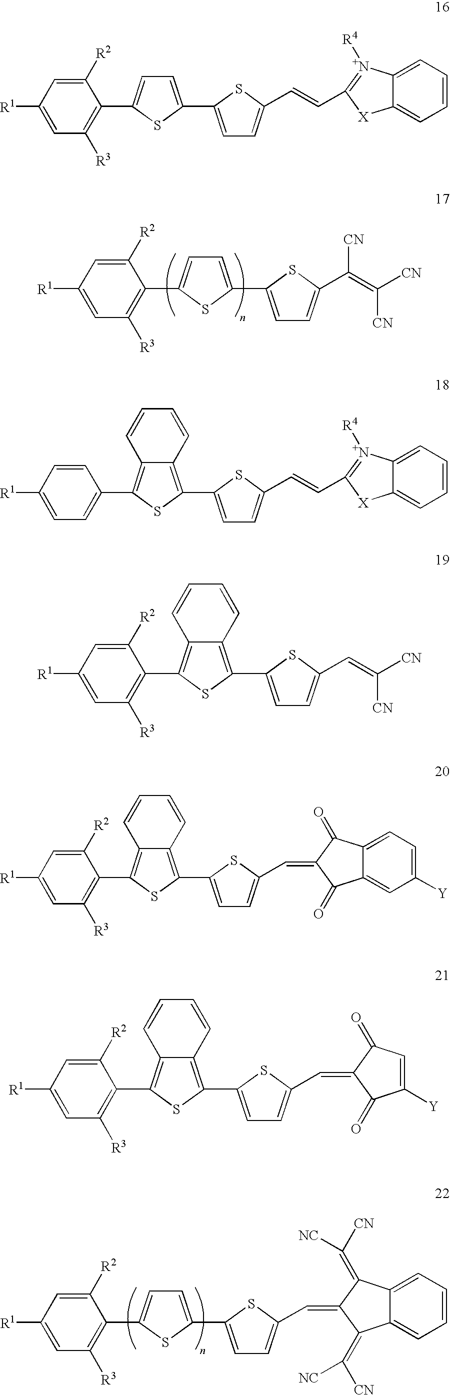

Also preferred are compounds of general formula 1a, which can be represented by structures 23-24:

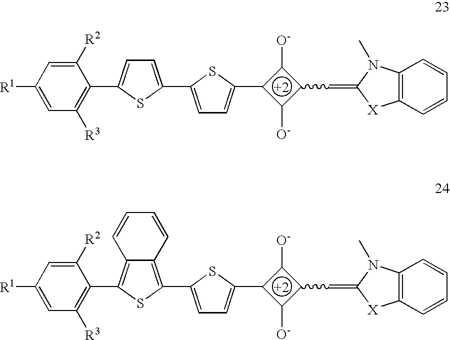

In the structures 16-24, R¹, R², and R³ can, independently of one another, stand for —OH, —NMe₂, —N(CH₂CH₂OH)₂, or hydrogen. There is at least one substituent R¹-R³ different from hydrogen. Independently on these substituents, R⁴ can be Me or —(CH₂)₃—SO₂O—, with the Me group requiring Cl⁻ as a counteranion. Also independently, X can stand for —C(Me₂)—, S, O, or NH, and Y can be either hydrogen or —COOH. In the formulae 17 and 22, n can be equal either 1, 2, or 3.

### Example 3

We have prepared the following detectable compounds:

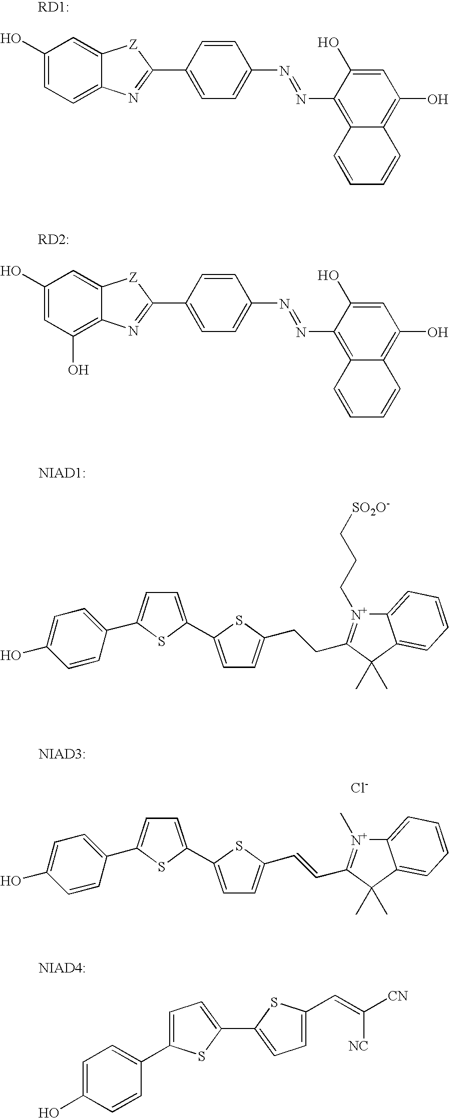

Table 3 indicates properties of the compounds.

TABLE 3
Properties of certain dye compounds (ND = Not determined)

| | Properties of compound | | |
|---|---|---|---|
| | Plaque | Enter CNS | Near-IR |
| | specificity | (BBB) | spectra |
| RD1 | Yes | Yes | No |
| RD2 | Yes | Yes | No |
| NIAD1 | Yes | Yes/No | Yes |
| NIAD3 | Yes | No | Yes |
| NIAD4 | Yes | Yes | No |
| NIAD6 | Yes | No | Yes |
| NIAD7 | Yes | Yes | No |
| NIAD9 | No | ND | Yes |
| NIAD10 | No | ND | Yes |

### Example 4

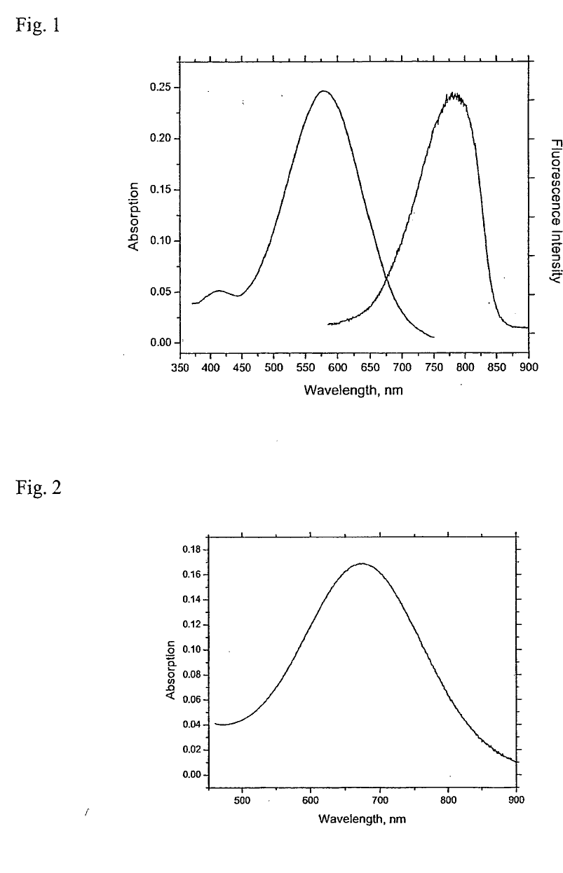

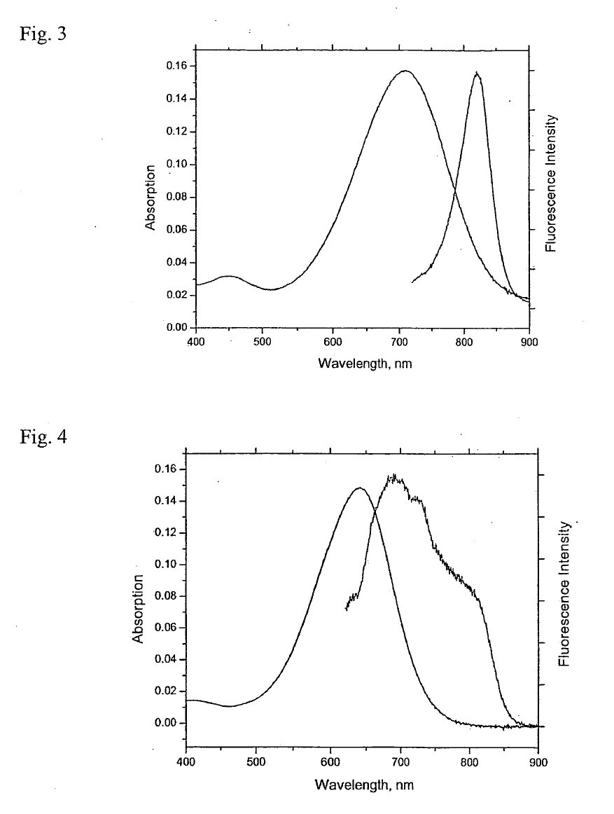

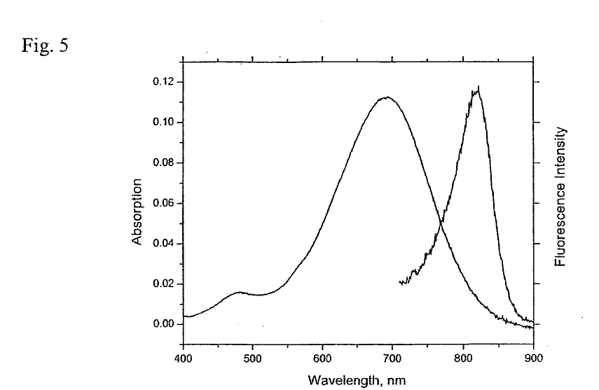

We have prepared the detectable compounds indicated as NIAD6, NIAD7, NIAD8, NIAD9, and NIAD10 were prepared and the absorption and/or emission spectra for each compound in methanol, DMSO, or THF solution were determined. The compounds and absorption and emission spectra for NIAD6, NIAD7, NIAD8, NIAD9, and NIAD10 are shown in FIGS. 1-5 respectively.

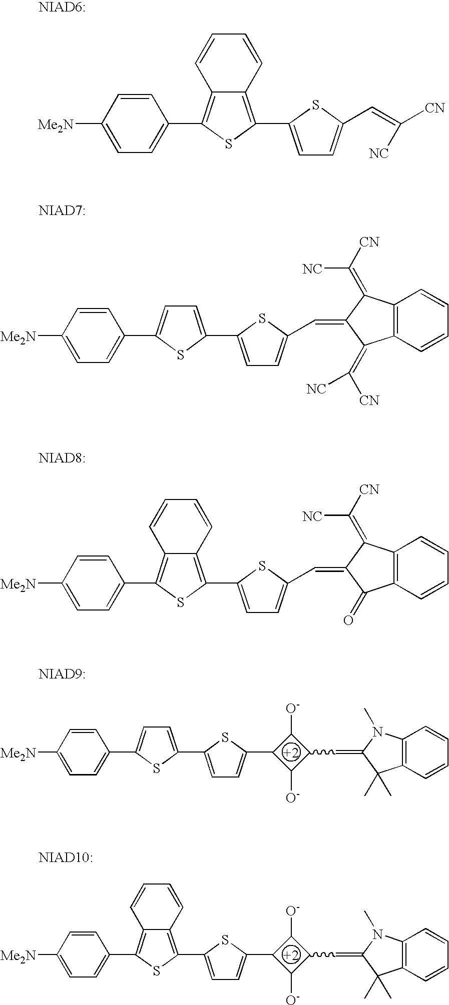

### Example 5

Structures were identified that represent a new class of dyes bearing a triazole functional group as the donor-subunit. This represents a novel approach for constructing modular dyes. Through judicious choice of the subunits the absorption and emission of the dyes could be readily tuned in a range between 300 and 700 mm.

1) The general structures of the new dyes included a triazole group (donor-subunit), hetero-aromatic bridge (polarizable bridge), and an electron-withdrawing group (EWG; also designated with R-group designations elsewhere herein):

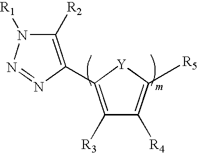

### (Compound 20)

In the structure, m can be equal to 1, 2, or 3 and R₅ is H or an electron withdrawing group (EWG). In some embodiments, Y was S. Examples of EWG include, but are not limited to: F, Br, C₁, CF₃, CN, CHO, CONH₂, COOH, COOR′, COCH₃, COR′, NO₂, CON(CH₃)₂, CONR′₂, COOCH₃, COOR′, SO₃H, SO₃R′, CCl₃, N′₄⁺, NR′₃⁺, NR′₄⁺ wherein R′ is a lower alkyl group other than CH₃,

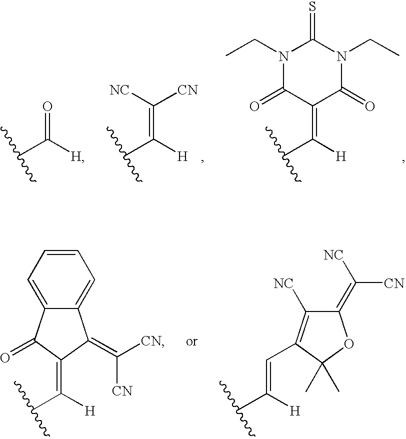

2) Examples of specific structures that have been synthesized and characterized for binding and imaging β-amyloid plaques include:

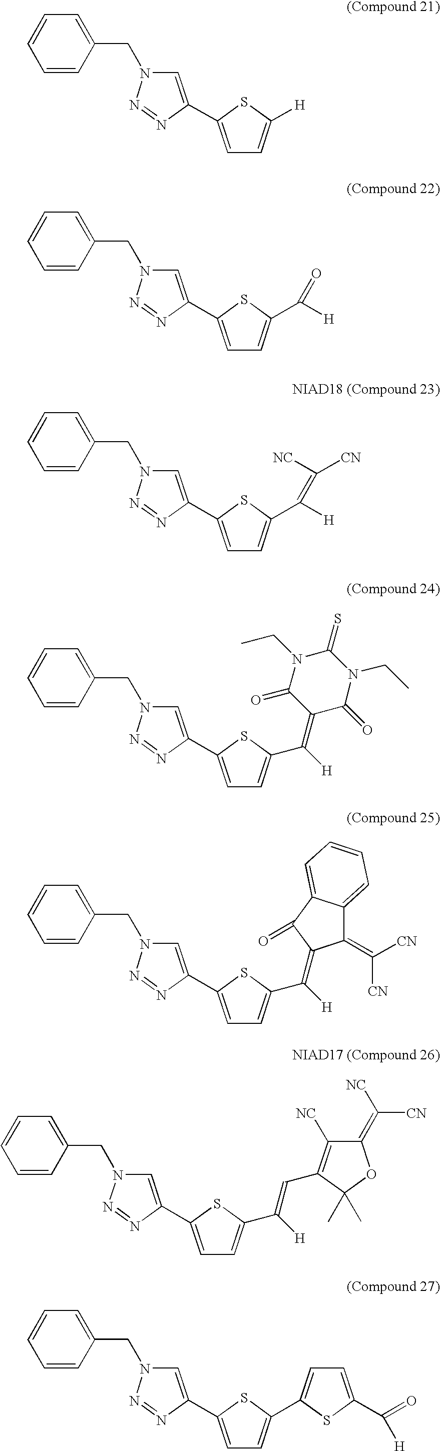

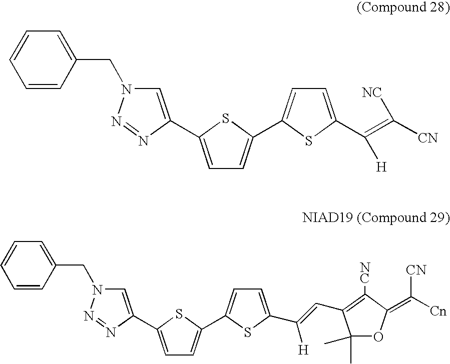

3) Spectral information for compounds based on compound 20:

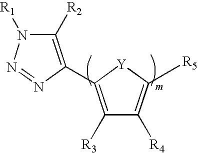

TABLE 4
Spectral information for compounds with H or Electron
withdrawing group indicated as 1-5.

| entry | λ_{ab} (nm) | λₑₘ (nm) |
|---|---|---|
| 1 | 270 | 334 |
| 2 | 324 | 387 |
| 3 | 398 | 456 |
| 4 | 485 | 594 |
| 5 | 548 | 657 |

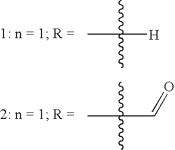

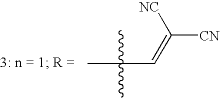

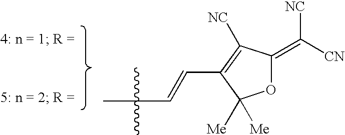

4) The following derivatives were tested for binding to amyloid-beta deposits (all showed weak binding):

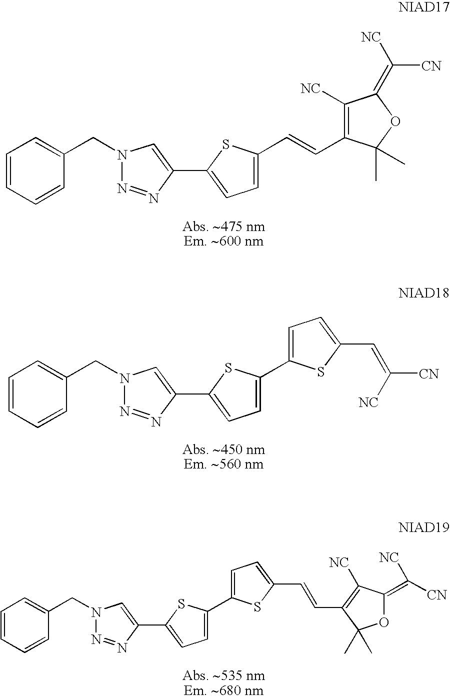

Other aspects of the invention will be clear to the skilled artisan and need not be repeated here. All patents, published patent applications and literature cited herein are incorporated by reference in their entirety.

While the invention has been described with respect to certain embodiments, it should be appreciated that many modifications and changes may be made by those of ordinary skill in the art without departing from the spirit of the invention. It is intended that such modification, changes and equivalents fall within the scope of the following claims.

We claim:

1. A compound of the formula: 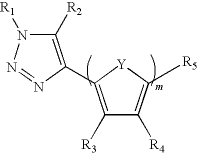
wherein
R₁-R₄ are independently: X, wherein X is F, Cl, Br or I, (CH₂)ₙOH wherein n=1, 2 or 3, CH₃, CF₃, CN, H, OHX, CHO, NH₂, CONH₂, OCOH, OH, SH, COOH, SnH₃, R′, R′OR′, R′OCH₃, CH₂X, R′X, OCH₂X, OR′, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃, SnR′₃ wherein R′ is a lower alkyl group other than CH₃, benzene, substituted benzene, toluene, substituted toluene, xylene, substituted xylene;
R₅ is independently H or an electron withdrawing group: X, wherein X is F, Br, C₁, CF₃, CN, CHO, CONH₂, COOH, COOR′, COCH₃, COR′, NO₂, CON(CH₃)₂, CONR′₂, COOCH₃, COOR′, SO₃H, SO₃R′, CCl₃, NH₄⁺, NR′₃⁺, NR′₄⁺ wherein R′ is a lower alkyl group other than CH₃, 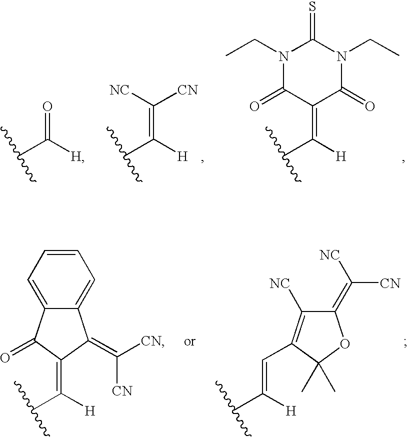
m is 1, 2, or 3; and
Y is independently S, O, or N.

2. The compound of claim 1 wherein Y is S.

3. The compound of claim 2 wherein R₁ is toluene.

4. The compound of claim 1 wherein R₂, R₃, and R₄ are H.

5. The compound of claim 1 wherein R₅ is an electron withdrawing group selected from the list of compounds consisting of:

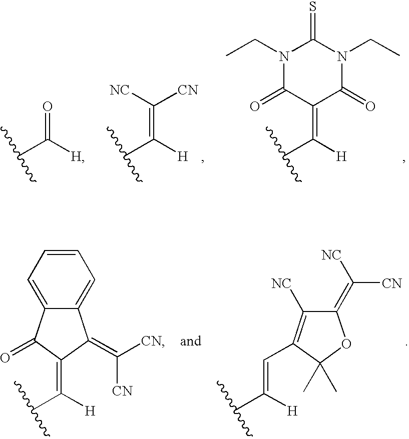

6. The compound of claim 1 wherein the compound is:

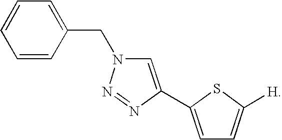

7. The compound of claim 1 wherein the compound is:

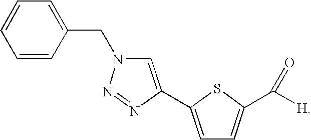

8. The compound of claim 1 wherein the compound is:

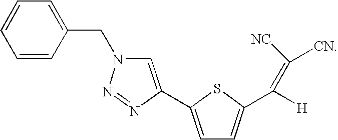

9. The compound of claim 1 wherein the compound is:

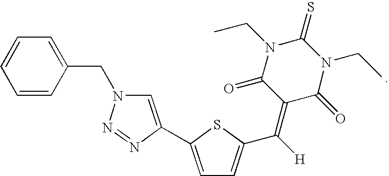

10. The compound of claim 1 wherein the compound is:

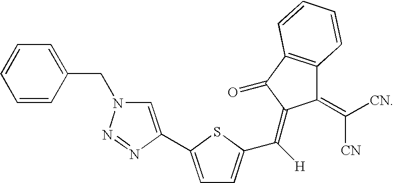

11. The compound of claim 1 wherein the compound is:

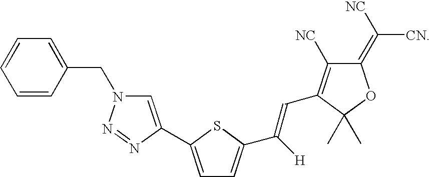

12. The compound of claim 1 wherein the compound is:

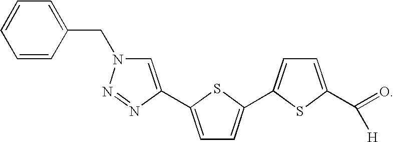

13. The compound of claim 1 wherein the compound is:

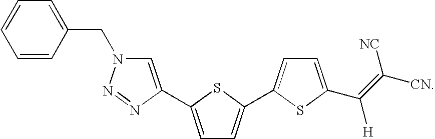

14. The compound of claim 1 wherein the compound is:

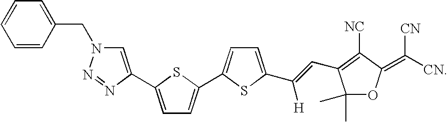

15. A compound of the formula: 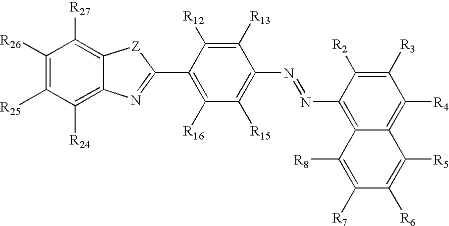
wherein
Z is S, NR′, NH or O;
and wherein each R₂-R₈, R₁₂, R₁₃, R₁₅, R₁₆ and R₂₄-R₂₇ are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

16. The compound of claim 15, wherein
R₂, R₄, and R₂₆ are OH.

17. The compound of claim 16, wherein the compound is:

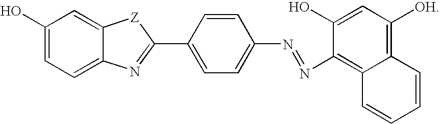

18. The compound of claim 15, wherein
R₂, R₄, R₂₄, and R₂₆ are OH.

19. The compound of claim 18, wherein the compound is:

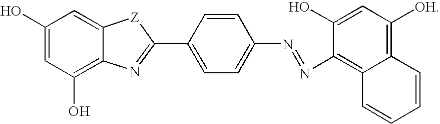

20. The compound of claim 15, wherein
Z is S;
R₅-R₈, R₁₂, R₁₃, R₁₅, 16, R₂₄, R₂₅, and R₂₇ are H;
R₂, R₃, R₄, and R₂₆ are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

21. The compound of claim 20, wherein:
R₂ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₃ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, SnH₃, R′, R′OR′, R′OCH₃, CH₂X, R′, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₄ and R₂₆ are H;
or
R₂ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′, OCH₂X, OR′, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₂₆ is X wherein X is F, Cl, Br or I, (CH₂)ₙOH wherein n=1, 2 or 3, CF₃, CN, H, HOH, OHX, COH, NH₂, CONH₂, OCOH, OH, SH, COOH, SnH₃, R′, R′OR′, R′OCH₃, CH₂X, R′, OCH₂X, OR′, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₃ and R₄ are H;
or
R₃ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, SnH₃, R′, R′OR′, R′OCH₃, CH₂X, R′, OCH₂X, OR′, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₄ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′, OCH₂X, OR′, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₂₆ is: X wherein X is F, Cl, Br or I, (CH₂)ₙOH wherein n=1, 2 or 3, CF₃, CN, H, HOH, OHX, COH, NH₂, CONH₂, OCOH, OH, SH, COOH, SnH₃, R′, R′OR′, R′OCH₃, CH₂X, R′, OCH₂X, OR′, COCH₃, COR′, N(CH₃)₂, NR′₂, NO₂, CON(CH₃)₂, CONR′₂, OCOCH₃, OCOR′, OR′, SCH₃, SR′, COOCH₃, COOR′, SnCH₃ or SnR′₃ wherein R′ is a lower alkyl group other than CH₃;
and R₂ is H;
or
R₄ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₂₆ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₂ and R₃ are H;
or
R₂ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₃, R₄ and R₂₆ are H;
or
R₄ is X wherein X is F, Cl, Br or I, (CH₂)ₙOH wherein n=1, 2 or 3, CH₃, CF₃, CN, H, HOH, OHX, COH, CONH₂, OCOH, SH, COOH, SnH₃, R′, R′OR′, R′OCH₃, CH₂X, R′X, OCH₂X, OR′X, COCH₃, COR′, N(CH₃)₂, NH₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′ 3 wherein R′ is a lower alkyl group other than CH₃;
and R₂, R₃ and R₂₆ are H;
or
R₂ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₂₆ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OR′, SCH₃, SR′, COOCH₃, COOR′, SnCH₃ or SnR′₃ wherein R′ is a lower alkyl group other than CH₃;
and R₃ and R₄ are H;
or
R₄ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₂, R₃ and R₂₆ are H;
or
R₂ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₂₆ is X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₃ and R₄ are H;
or
R₄ is: X wherein X is F, Cl, Br or I, (CH₂)ₙOH wherein n=1, 2 or 3, CH₃, CF₃, CN, H, HOH, OHX, COH, NH₂, CONH₂, OCOH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₂₆ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R¹ is a lower alkyl group other than CH₃;
and R2 and R3 are H.

22. The compound of claim 20 wherein
R₂ is: X wherein X is F, Cl, Br or I, (CH₂)ₙOH wherein n=1, 2 or 3, CH₃, CF₃, CN, H, HOH, OHX, COH, CONH₂, OCOH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₃ is: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₄ is: X wherein X is F, Cl, Br or I, (CH₂)ₙOH wherein n=1, 2 or 3, CH₃, CF₃, CN, H, HOH, OHX, COH, CONH₂, OCOH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₂₆ is: X wherein X is F, Cl, Br or I, (CH₂)ₙOH wherein n=1, 2 or 3, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, NR′₂, NO₂, CON(CH₃)₂, CONR′₂, OCOCH₃, OCOR′, OR′, SCH₃, SR′, COOCH₃, COOR′, SnCH₃ or SnR′₃ wherein R′ is a lower alkyl group other than CH₃.

23. The compound of claim 20, wherein if R₂ is OH, R₃ cannot be CO₂H.

24. The compound of claim 20, wherein if R₃ is CO₂H, R₂ cannot be OH.

25. The compound of claim 20, wherein if R₂ is OH, R₂₆ cannot be CH₃.

26. The compound of claim 20, wherein if R₂₆ is CH₃, R₂ cannot be OH.

27. The compound of claim 20, wherein if R₃ is CO₂H, R₄ and R₂₆ cannot be OH and CH₃, respectively.

28. The compound of claim 20, wherein if R₄ is OH, R₃ and R₂₆ cannot be CO₂H and CH₃, respectively.

29. The compound of claim 20, wherein if R₂₆ is CH₃, R₃ and R₄ cannot be CO₂H and OH, respectively.

30. The compound of claim 20, wherein if R₄ is OH, R₂₆ cannot be OCH₃.

31. The compound of claim 20, wherein if R₂₆ is OCH₃, R₄ cannot be OH.

32. The compound of claim 20, wherein R₂ is not NH₂.

33. The compound of claim 20, wherein R₄ is not OH.

34. The compound of claim 20, wherein if R₂ is NH₂, R₂₆ cannot be OCH₃.

35. The compound of claim 20, wherein if R₂₆ is OCH₃, R₂ cannot be NH₂.

36. The compound of claim 20, wherein R₄ is not NH₂.

37. The compound of claim 20, wherein if R₂ is NH₂, R₂₆ cannot be N(CH₃)₂.

38. The compound of claim 20, wherein if R₂₆ is N(CH₃)₂, R₂ cannot be NH₂.

39. The compound of claim 20, wherein if R₄ is OH, R₂₆ cannot be N(CH₃)₂.

40. The compound of claim 20, wherein if R₂₆ is N(CH₃)₂, R₄ cannot be OH.

41. The compound of claim 15, wherein
Z is O;
R₃, R₅-R₈, R₁₂, R₁₃, R₁₅, R₁₆, R₂₄, R₂₆, and R₂₇ are H;
R₂, R₄, and R₂₅ are independently X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

42. The compound of claim 23, wherein:
R₄ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₂ and R₂₅ are H;
or
R₂ is: X wherein X is F, Cl, Br or I, (CH₂)ₙOH wherein n=1, 2 or 3, CH₃, CF₃, CN, H, HOH, OHX, COH, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
R₂₅ is: X wherein X is F, Cl, Br or I, (CH₂)ₙOH wherein n=1, 2 or 3, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃;
and R₄ is H.

43. The compound of claim 41, wherein R₄ is not NH₂.

44. The compound of claim 41, wherein if R₂ is NH₂, R₂₅ cannot be CH₃.

45. The compound of claim 41, wherein if R₂₅ is CH₃, R₂ cannot be NH₂.

46. A compound of the formula: 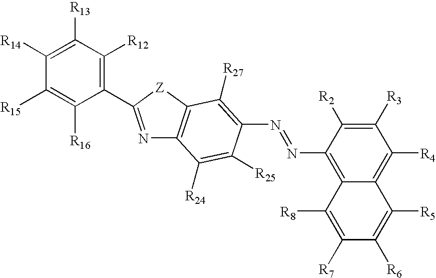
wherein
Z is S, NR′, NH or 0;
and wherein each R₂-R₈, R₁₂-R₁₆ and R₂₄, R₂₅, and R₂₇ are independently X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

47. A compound of claim 46, wherein
Z is S;
R₂, R₄, R₂₆ are OH;
R₂₄ is OH or H; and
R₅₋₈, R₁₂, R₁₃, R₁₅, R₁₆, R₂₅ and R₂₇ are H.

48. A compound of claim 46, wherein
Z is S;
R₂, R₄, R₁₄, are OH;
R₁₆ is OH or H;
and R₅₋₈, R₁₂₋₁₃, R₁₅ and R₂₄, R₂₅, R₂₇ are H.

49. A compound of the formula: 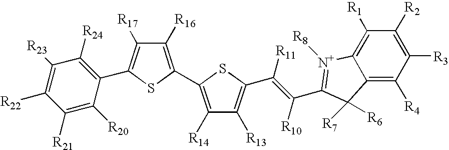
wherein
each R₁-R₄, R₆-R₈, R₁₀-R₁₁, R₁₃-R₁₄, R₁₆-R₁₇, and R₂₀-R₂₄ are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, N′R₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃, and wherein R₈ can be (CH₂)₃SO₂O.

50. The compound of claim 49, wherein
R₂₂, R₂₄, and R₂₀ are independently —OH, —NMe₂, —N(CH₂CH₂OH)₂, or hydrogen; there is at least one substituent R₂₂, R₂₄, and R₂₀ different from hydrogen; R₈ is independently Me or —(CH₂)₃—SO₂⁻, with the Me group requiring Cl⁻ as a counteranion; and X is —C(Me₂)—, S, O, or NH.

51. The compound of claim 49, wherein the compound is:

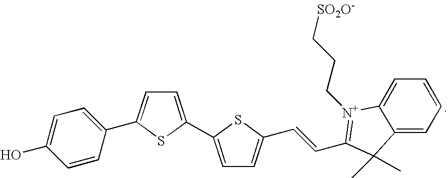

52. The compound of claim 49, wherein the compound is:

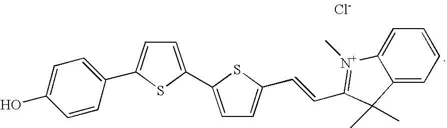

53. A compound of the formula: 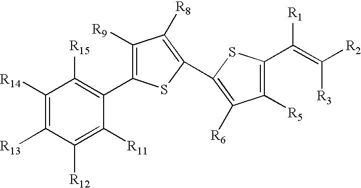
wherein
each R₁-R₃, R₅-R₆, R₈-R₉, and R₁₁-R₁₅, are independently: X wherein X is F, Cl, Br or I, (CH₂)ₙOH wherein n=˜1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃; and m is one, two, or three.

54. The compound of claim 53, wherein
R₁₃, R₁₅, and R₁₁, are independently —OH, —NMe₂, —N(CH₂CH₂OH)₂, or hydrogen; there is at least one substituent R₁₃, R₁₅, and R₁₁ different from hydrogen; and n can be one, two, or three.

55. The compound of claim 53, wherein
R₁₃, R₁₅, and R₁₁ are independently —OH, —NMe₂, —N(CH₂CH₂OH)₂, or hydrogen; there is at least one substituent R₁₃, R₁₅, and R₁₁ different from hydrogen; m can be one, two or three; and R₁, R₂, and R₃ are CN.

56. The compound of claim 53, wherein the compound is:

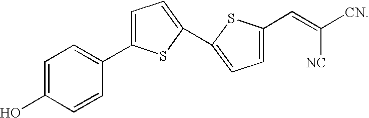

57. A compound of formula: 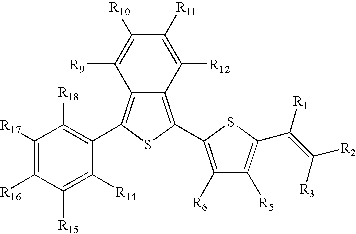
wherein
each R₁-R₃, R₅-R₆, R₉-R₁₂, and R₁₄-R₁₈, are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

58. The compound of claim 57, wherein
R₁₆, R₁₈, and R₁₄ are independently —OH, —NMe₂, —N(CH₂CH₂OH)₂, or hydrogen; and there is at least one substituent R₁₆, R₁₈, and R₁₄ different from hydrogen.

59. The compound of claim 57, wherein the compound is:

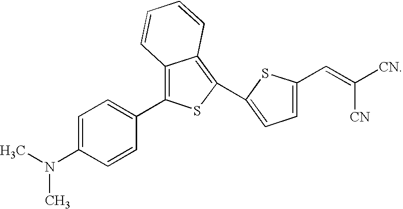

60. A compound of the formula: 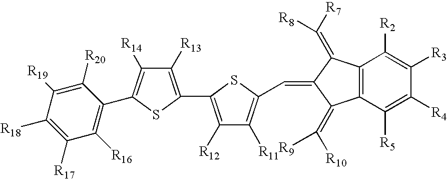
wherein
each R₂-R₅, R₇-R₁₀, R₁₁-R₁₄, and R₁₆-R₂₀, are independently: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃, and m is one, two, or three.

61. The compound of claim 60, wherein
R₁₈, R₂₀, and R₁₆ are independently —OH, —NMe₂, —N(CH₂CH₂OH)₂, or hydrogen; there is at least one substituent R₁₈, R₂₀, or R₁₆ different from hydrogen; and m can be one, two, or three.

62. The compound of claim 60, wherein the compound is:

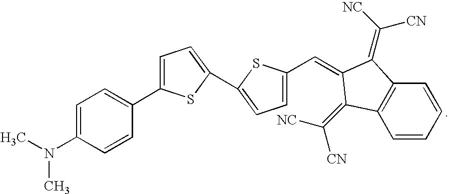

63. A compound of the formula: 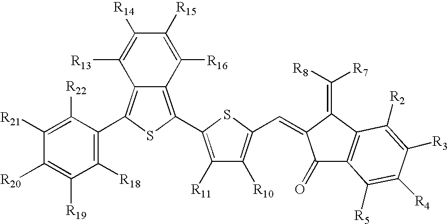
wherein
each R₂-R₅, R₇-R₈, R₁₀-R₁₁, R₁₃-R₁₆, and R₁₈-R₂₂, are: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

64. The compound of claim 63, wherein the compound is:

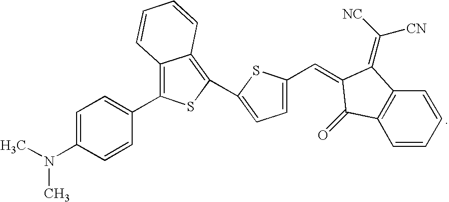

65. A compound of the formula: 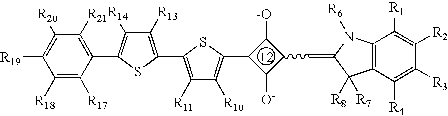
wherein
each R₁-R₄, R₆-R₈, R₁₀-R₁₁, R₁₃-R₁₄, and R₁₇-R₂₁, are: X wherein X is F, Cl, Br or I, (CH₂)ₙOH wherein n−1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

66. The compound of claim 65, wherein the compound is:

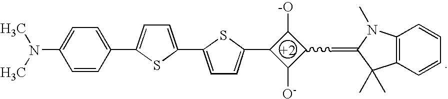

67. A compound of the formula: 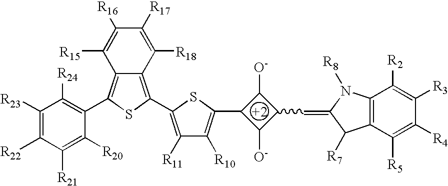
wherein
each R₂-R₅, R₇-R₈, R₁₀-R₁₁, R₁₅-R₁₈, and R₂₀-R₂₄, are: X wherein X is F, Cl, Br or I, (CH₂)ₙOH wherein n=1, 2 or 3, CH₃, CF₃, CN, H, HOH, OHX, COH, NH₂, CONH₂, OCOH, OH, SH, COOH, SnH₃, R′, R′OR′, R′OCH₃, CH₂X, R′X, OCH₂X, OR′X, COCH₃, COR′, N(CH₃)₂, NR′₂, NO₂, CON(CH₃)₂, CONR′₂, OCOCH₃, OCOR′, OCH₃, OR′, SCH₃, SR′, COOCH₃, COOR′, SnCH₃ or SnR′₃ wherein R′ is a lower alkyl group other than CH₃.

68. The compound of claim 67, wherein the compound is:

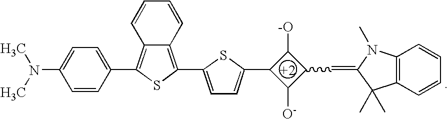

69. A compound of the formula: 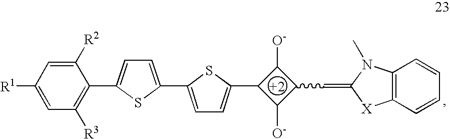
wherein, R¹, R², and R³ are independently-OH, —NMe₂, —N(CH₂CH₂OH)₂, or hydrogen; there is at least one substituent R¹-R³ different from hydrogen; R⁴ is independently Me or —CH₂)₃—SO₂O—, with the Me group requiring Cl⁻ as a counteranion; and X is —C(Me₂), S, O, or NH.

70. A compound of the formula: 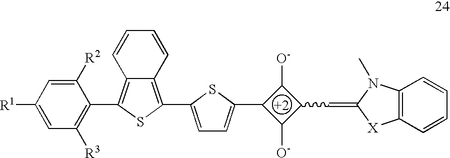
wherein, R¹, R², and R³ are independently —OH, —NMe₂, —N(CH₂CH₂OH)₂, or hydrogen; there is at least one substituent R¹-R³ different from hydrogen; R⁴ is independently Me or —(CH₂)₃—SO₂O—, with the Me group requiring Cl⁻ as a counteranion; and X is —C(Me₂)—, S, O, or NH.

71. A compound of formula: 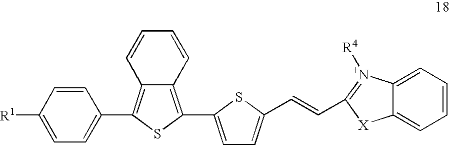
wherein R′ is independently —OH, —NMe₂, —N(CH₂CH₂OH)₂, or hydrogen; R⁴ is independently Me or —CH₂)₃—SO₂O—, with the Me group requiring Cl⁻ as a counteranion; and X is —C(Me₂)—, S, O, or NH.

72. A compound of formula: 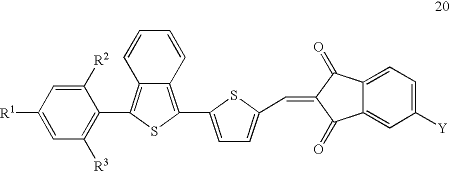
wherein R¹, R², and R³ are independently —OH, —NMe₂, —N(CH₂CH₂OH)₂, or hydrogen; there is at least one substituent R¹-R³ different from hydrogen; and Y is either hydrogen or —COOH.

73. A compound of formula: 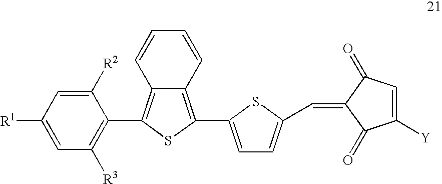
wherein, R¹, R², and R³ are independently —OH, —NMe₂, —N(CH₂CH₂OH)₂, or hydrogen; there is at least one substituent R¹-R³ different from hydrogen; and Y is either hydrogen or —COOH.

74-77. (canceled)

78. A method for in vivo imaging of amyloid deposits comprising:
administering a detectable amount of a compound of claim 1, to a subject suspected of having amyloid deposits, and
detecting the compound to image the amyloid deposit.

79. The method of claim 78, wherein the amyloid deposit is located in the brain of a subject.

80. The method of claim 79, wherein the subject is suspected of having a disease or syndrome that is: Alzheimer's Disease, familial Alzheimer's Disease, Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, inclusion body myositis, multiple sclerosis, or homozygotes for the apolipoprotein E4 allele.

81. The method of claim 78, wherein the detecting is infrared imaging, multiphoton imaging, gamma imaging, magnetic resonance imaging or magnetic resonance spectroscopy.

82. The method of claim 81, wherein the detecting is infrared imaging.

83. The method of claim 81, wherein the detecting is gamma imaging, and the gamma imaging is either PET or SPECT.

84. The method of claim 78, wherein the detecting is done by microscopy.

85. The method of claim 78, wherein the pharmaceutical composition is administered by intravenous injection.

86. The method of claim 79, wherein the ratio of (i) binding of the compound to a brain area other than the cerebellum to (ii) binding of the compound to the cerebellum, in the subject, is compared to the ratio of (i) to (ii) in normal subjects.

87. (canceled)

88. The compound of claim 87, wherein the detectable label is a radiolabel, fluorescent label, enzyme, or chemiluminescent molecule.

89. A method of evaluating a treatment for an amyloid-associated disorder comprising:
administering a first detectable amount of a compound of claim 1 to a subject undergoing treatment for an amyloid-associated disorder to obtain a first level of binding of the compound to amyloid in the subject,
detecting the compound bound to amyloid to determine the first level of binding of the compound,
administering a second detectable amount of the compound, wherein the second administration is at a time subsequent to the first administration, to obtain a second level of binding of the compound to amyloid in the subject,
detecting the compound bound to amyloid to determine the second level of binding of the compound, and
comparing the first level of binding with the second level of binding as an indication of the effectiveness of the treatment on the level of amyloid in the subject.

90. The method of claim 89, wherein the amyloid-associated disorder is Alzheimer's Disease, familial Alzheimer's Disease, Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, inclusion body myositis, multiple sclerosis, or homozygotes for the apolipoprotein E4 allele.

91. A method of selecting a treatment for an amyloid-associated disorder in a subject comprising:
administering a detectable amount of a compound of claim 1 to a subject to obtain a level of binding of the compound to amyloid,
detecting the compound bound to amyloid to determine the level of binding of the compound, and
selecting the treatment for the amyloid-associated disorder based at least in part on the level of binding obtained.

92. The method of claim 91, wherein the amyloid-associated disorder is Alzheimer's Disease, familial Alzheimer's Disease, Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, inclusion body myositis, multiple sclerosis, or homozygotes for the apolipoprotein E4 allele.

93. A method for determining regression, progression or onset of an amyloid-associated disorder comprising;
administering a detectable amount of a compound of claim 1 to a subject to obtain a level of binding of the compound to amyloid,
detecting the compound bound to amyloid to determine the level of binding of the compound, and
comparing the level of binding of the compound to a control level of binding of the compound as a indication of regression, progression or onset of the condition.

94. The method of claim 93, wherein the amyloid-associated disorder is Alzheimer's Disease, familial Alzheimer's Disease, Down's syndrome, cerebrovascular amyloidosis (Cerebral Amyloid Angiopathy), Hereditary Amyloidosis with Cerebral Hemorrhage of the Dutch Type (HCHWA-D), Familial British Dementia, vascular dementia, inclusion body myositis, multiple sclerosis, or homozygotes for the apolipoprotein E4 allele.


SEQUENCE LISTING (2 sequences)

SEQ ID NO: 1 - protein, 5 aa, Artificial sequence
  Caspase Inhibitor
XDVAX

SEQ ID NO: 2 - protein, 4 aa, Artificial sequence
  Caspase Inhibitor
XEVX